SEQUENCE LISTING (4 sequences)

SEQ ID NO: 1 - protein, 195 aa, Homo sapiens
MGRARDAILDALENLTAEELKKFKLKLLSVPLREGYGRIPRGALLSMDALDLTDKLVSFYLETYGAELTANVLRDMGLQEMAGQLQAATHQGSGAAPAGIQAPPQSAAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 2 - protein, 713 aa, Homo sapiens
MQPRTPLVLCVLLSQVLLLTSAEDLDCTPGFQQKVFHINQPAEFIEDQSILNLTFSDCKGNDKLRYEVSSPYFKVNSDGGLVALRNITAVGKTLFVHARTPHAEDMAELVIVGGKDIQGSLQDIFKFARTSPVPRQKRSIVVSPILIPENQRQPFPRDVGKVVDSDRPERSKFRLTGKGVDQEPKGIFRINENTGSVSVTRTLDREVIAVYQLFVETTDVNGKTLEGPVPLEVIVIDQNDNRPIFREGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIMIDDKNDHSPKFTKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTRQEDLSVGSVLLTVNATDPDSLQHQTIRYSVYKDPAGWLNINPINGTVDTTAVLDRESPFVDNSVYTALFLAIDSGNPPATGTGTLLITLEDVNDNAPFIYPTVAEVCDDAKNLSVVILGASDKDLHPNTDPFKFEIHKQAVPDKVWKISKINNTHALVSLLQNLNKANYNLPIMVTDSGKPPMTNITDLRVQVCSCRNSKVDCNAAGALRFSLPSVLLLSLFSLACL

SEQ ID NO: 3 - DNA, 879 bp, Homo sapiens
gagggcgcgatcctggcgtcccccgacggcctggggccccaatccagaggcctgggtgggaggggaccaagggtgtagtaaggaagcgccttttgctggagggcaacggaccggggcggggagtcgggagaccagagtgggaggaaggcggggagtccaggttccgccccggagccgacttcctcctggtcggcggctgcagcggggtgagcggcggcagcggccggggatcctggagccatggggcgcgcgcgcgacgccatcctggatgcgctggagaacctgaccgccgaggagctcaagaagttcaagctgaagctgctgtcggtgccgctgcgcgagggctacgggcgcatcccgcggggcgcgctgctgtccatggacgccttggacctcaccgacaagctggtcagcttctacctggagacctacggcgccgagctcaccgctaacgtgctgcgcgacatgggcctgcaggagatggccgggcagctgcaggcggccacgcaccagggcctgcactttatagaccagcaccgggctgcgcttatcgcgagggtcacaaacgttgagtggctgctggatgctctgtacgggaaggtcctgacggatgagcagtaccaggcagtgcgggccgagcccaccaacccaagcaagatgcggaagctcttcagtttcacaccagcctggaactggacctgcaaggacttgctcctccaggccctaagggagtcccagtcctacctggtggaggacctggagcggagctgaggctccttcccagcaacactccggtcagcccctggcaatcccaccaaatcatcctgaatctgatctttttatacacaatatacgaaaagccagcttgaaaaaaaaaaa

SEQ ID NO: 4 - DNA, 3842 bp, Homo sapiens
gggaagttggctggctggcgaggcagagcctctcctcaaagcctggctcccacggaaaatatgctcagtgcagccgcgtgcatgaatgaaaacgccgccgggcgcttctagtcggacaaaatgcagccgagaactccgctcgttctgtgcgttctcctgtcccaggtgctgctgctaacatctgcagaagatttggactgcactcctggatttcagcagaaagtgttccatatcaatcagccagctgaattcattgaggaccagtcaattctaaacttgaccttcagtgactgtaagggaaacgacaagctacgctatgaggtctcgagcccatacttcaaggtgaacagcgatggcggcttagttgctctgagaaacataactgcagtgggcaaaactctgttcgtccatgcacggaccccccatgcggaagatatggcagaactcgtgattgtcggggggaaagacatccagggctccttgcaggatatatttaaatttgcaagaacttctcctgtcccaagacaaaagaggtccattgtggtatctcccattttaattccagagaatcagagacagcctttcccaagagatgttggcaaggtagtcgatagtgacaggccagaaaggtccaagttccggctcactggaaagggagtggatcaagagcctaaaggaattttcagaatcaatgagaacacagggagcgtctccgtgacacggaccttggacagagaagtaatcgctgtttatcaactatttgtggagaccactgatgtcaatggcaaaactctcgaggggccggtgcctctggaagtcattgtgattgatcagaatgacaaccgaccgatctttcgggaaggcccctacatcggccacgtcatggaagggtcacccacaggcaccacagtgatgcggatgacagcctttgatgcagatgacccagccaccgataatgccctcctgcggtataatatccgtcagcagacgcctgacaagccatctcccaacatgttctacatcgatcctgagaaaggagacattgtcactgttgtgtcacctgcgctgctggaccgagagactctggaaaatcccaagtatgaactgatcatcgaggctcaagatatggctggactggatgttggattaacaggcacggccacagccacgatcatgatcgatgacaaaaatgatcactcaccaaaattcaccaagaaagagtttcaagccacagtcgaggaaggagctgtgggagttattgtcaatttgacagttgaagataaggatgaccccaccacaggtgcatggagggctgcctacaccatcatcaacggaaaccccgggcagagctttgaaatccacaccaaccctcaaaccaacgaagggatgctttctgttgtcaaaccattggactatgaaatttctgccttccacaccctgctgatcaaagtggaaaatgaagacccactcgtacccgacgtctcctacggccccagctccacagccaccgtccacatcactgtcctggatgtcaacgagggcccagtcttctacccagaccccatgatggtgaccaggcaggaggacctctctgtgggcagcgtgctgctgacagtgaatgccacggaccccgactccctgcagcatcaaaccatcaggtattctgtttacaaggacccagcaggttggctgaatattaaccccatcaatgggactgttgacaccacagctgtgctggaccgtgagtccccatttgtcgacaacagcgtgtacactgctctcttcctggcaattgacagtggcaaccctcccgctacgggcactgggactttgctgataaccctggaggacgtgaatgacaatgccccgttcatttaccccacagtagctgaagtctgtgatgatgccaaaaacctcagtgtagtcattttgggagcatcagataaggatcttcacccgaatacagatcctttcaaatttgaaatccacaaacaagctgttcctgataaagtctggaagatctccaagatcaacaatacacacgccctggtaagccttcttcaaaatctgaacaaagcaaactacaacctgcccatcatggtgacagattcagggaaaccacccatgacgaatatcacagatctcagggtacaagtgtgctcctgcaggaattccaaagtggactgcaacgcggcaggggccctgcgcttcagcctgccctcagtcctgctcctcagcctcttcagcttagcttgtctgtgagaactcctgacgtctgaagcttgactcccaagtttccatagcaacaggaaaaaaaaaaatctatccaaatctgaagattgcggtttacagctatcgaacttcacaactaggcctcaattgttccggttttttattttctttacaatttcacttagtctgtacttcatcattttgacagcatcttcctccctcctttaattaatggaatcttctgaattttccctgaatgtttaaagatcatgacatatgacttgatcttctgggagcaggaacaatgactactttttctggtgtgttaacatgtcgctagccagtgctccaggcacccagctttgtctgtgggttagtattggtgtatgtatgagtatctgtatgtatatatacacggtatttatagagagagactatcctggagaagcctcgttttgatgccattcttccttgcaaggttaagcaaggtgggtggaaactaagacacctgaaccctccagggcctcccgcatcaaggtcagcatgaggacagaccacagagctgtcacttttgctccgaagctacttctccactgtcccgttcagtctgaatgctgccacaaccagccaggcaggtccacagagagggagagcagagaaagaagtcctttctctttattgagttcgaggactacaaccaatttacactgccatctgatgccgtgatcctgagccaaggaggtgaggagcagagcaggcaatttcaccaccaaatgccaagaaaagggctgacattttctttcatgggcaccaacctgcatttgtatgtgtcccgaatccacagtcgtactgattctaatggggacacagatcatggtagagaatctctccctcctcagtaaatgtacaactgcacctgtcatcatggaggtcatacatgcatacaaagaggtgtacaggtaccatcttgtatacacatatatacccacatgtacagacatacatttatgcacattcacgctgtttgtttcatatatacaggcataaaatagagtaaatacaggtagttttaaaagtacccttttgtgtgaattgactaccgttgtttgcaaacccgaaaataaaagacgttcattatgtatgaaaagtaactgatttgtattctgtgagcatgtaaaagcggaaagttagtgcttgttctaagattaccttcttgttgataaaccataaatgaatcatcaaagctcacaccaaatttttctatcaaataaaactagtgacagcttgtggctttttattagagctcgccacgaactagggtaaggtgagtgtcttagcatattttaatgcagttgcttactaaaggttttaaccgcacatgcacacacacacgctttcttatgcaatctatgtttgcacttgtgctttcagttagccttctgtaggaagtagaagtcatatgttgtctttgttgtagtgaaattatacagatagagttccatatattgtatttgtttcaatggtaaatccttttggaacatatagaatgcagagatttttttttccattaaaataaatgggtattggtggttaaaaaaaaaaaaaaaaaa

(19) United States
(12) Patent Application Publication
(10) Pub. No.: US 2010/0297067 A1
(43) Pub. Date: Nov. 25, 2010

(54) METHYLATION MARKERS FOR PROSTATE CANCER AND METHODS OF USE

(75) Inventors: James Gordon Herman, Lutherville, MD (US); Joshi Alumkal, Portland, OR (US); Zhe Zhang, Owings Mills, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/600,413

(22) PCT Filed: May 14, 2008
(86) PCT No.: PCT/US2008/006179

§ 371 Date: Aug. 9, 2010

Related U.S. Application Data
(60) Provisional application No. 60/930,147, filed on May 14, 2007.

(51) Int. Cl. A61K 38/20; C12Q 1/68; G01N 33/50; A61P 35/00

(52) U.S. Cl. 424/85.2; 435/6; 436/94

(57) ABSTRACT

The present invention provides methods for identifying prostate cancer by detecting nucleic acid methylation of one or more genes in one or more samples. The invention further relates to DNA methylation as a predictor of prostate cancer recurrence and patient prognosis.

### RELATED APPLICATIONS

This application claims the benefit of U.S. Provisional Application No. 60/930,147, filed May 14, 2007. The entire contents of the aforementioned application are hereby incorporated herein by reference.

### FIELD OF THE INVENTION

The present invention relates to the use of nucleic acid methylation and methylation profiles to detect prostate cancer and in particular the recurrence of prostate cancer. The invention relates to methods for identifying a methylation profile that is independently associated with an increased risk of recurrence of prostate cancer. The method is of particular use in patients who have undergone radical prostatectamies. The invention further relates to DNA methylation as a predictor of disease recurrence and patient prognosis, specifically in patients suffering from prostate cancer.

### BACKGROUND OF THE INVENTION

Prostate cancer is the most common malignancy (33% of all cancers) and the third leading cause of cancer related mortality (9% of cancer deaths) in men in the United States (Walczak and Carducci, (2007) Mayo Clin Proc. 82:243-249). While some types of prostate cancer grow slowly and may need minimal or no treatment, other types are aggressive and can spread quickly.

Since the advent of prostate specific antigen (PSA) testing, more cancers are now detected at an earlier stage making “definitive” therapy with radiation or surgery more common (2, 3). Despite this stage shift and increase in primary treatment, both biochemical (PSA) and disease recurrences remain problematic (4). Several clinico-pathologic scoring systems have been developed to better identify prostate cancer patients at greatest risk of recurrence after surgery including the Rd risk score, which was developed using a modeling cohort of 904 patients from Johns Hopkins and a validation cohort of 901 patients from the Mayo Clinic, and the Kaftan nomograms (5, 6). However, 20 percent of patients who are defined as low-risk by these criteria still recur (5).

Prostate cancer is a disease whose complexity continues to unfold. Genetic events such as gene deletion, for example deletions of PTEN and NKX3.1, and gene fusions involving TMPRSS and ETS transcription factors, and somatic telomere shortening are associated with prostate tumorigenesis and/or aggressiveness (7-11). There is also strong evidence for the role of DNA methylation-induced gene silencing in the pathogenesis of prostate cancer (12-17).

DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. See, J. G. Herman, Seminars in Cancer Biology, 9: 359-67, 1999. DNA methylation plays an important role in determining gene expression. By turning genes off that are not needed, DNA methylation is an essential control mechanism for the normal development and functioning of organisms. Alternatively, abnormal DNA methylation is one of the mechanisms underlying the changes observed with the development of many cancers. However, a precise role for, and the significance of, abnormal DNA methylation in human tumorigenesis has not been well established.

Loss of gene function is cancer can occur by both genetic and epigenetic mechanisms. The best-defined epigenetic alteration of cancer genes involves DNA methylation of clustered CpG dinucleotides, or CpG islands, in promoter regions associated with the transcriptional inactivation of the affected genes. CpG islands are short sequences rich in the CpG dinucleotide, and can be found in the 5′ region of about half of all human genes. Methylation of cytosine within 5′ CGIs is associated with loss of gene expression and has been seen in a number of physiological conditions, including X chromosome inactivation and genomic imprinting. Aberrant methylation of CpG islands has been detected in genetic diseases such as the fragile-X syndrome, in aging cells and in neoplasia. About half of the tumor suppressor genes which have been shown to be mutated in the germline of patients with familial cancer syndromes have also been shown to be aberrantly methylated in some proportion of sporadic cancers, including Rb, VHL, p16, hMLH1, and BRCA1 (reviewed in Baylin, et al, Adv. Cancer Res. 72:141-196 1998). Methylation of tumor suppressor genes in cancer is usually associated with (1) lack of gene transcription and (2) absence of coding region mutation. Thus CpG island methylation can serve as an alternative mechanism of gene inactivation in cancer.

Although the phenomenon of gene methylation has attracted the attention of cancer researchers for some time, its true role in the progression of human cancers is just now being recognized. In normal cells, methylation occurs predominantly in regions of DNA that have few CG base repeats, while CpG islands, regions of DNA that have long repeats of CG bases, remain non-methylated. Gene promoter regions that control protein expression are often CpG island-rich. Aberrant methylation of these normally non-methylated CpG islands in the promoter region causes transcriptional inactivation or silencing of certain tumor suppressor expression in human cancers.

Genes that are methylated in tumor cells are strongly specific to the tissue of origin of the tumor. Molecular signatures of cancers of all types can be used to improve cancer detection, the assessment of cancer risk and response to therapy. Promoter methylation events provide some of the most promising markers for such purposes.

Cancer treatments, in general, have a higher rate of success if the cancer is diagnosed early, and treatment is started earlier in the disease process. A relationship between improved prognosis and stage of disease at diagnosis can be seen across a majority of cancers. Identification of the earliest changes in cells associated with cancer is thus a major focus in molecular cancer research. Diagnostic approaches based on identification of these changes in specific genes may allow implementation of early detection strategies and novel therapeutic approaches. Targeting these early changes will lead to more effective cancer treatment.

Patients who present with locally advanced, unresectable prostate tumors or metastatic disease usually progress to hormone-refractory disease for which curative systemic therapies are lacking. Further, affected individuals often are elderly, with limited tolerance for conventional cytotoxic chemotherapies.

Accordingly, there is a need in the art for improved methods of detection of prostate cancer, and in particular, for improved methods of detection of prostate cancer that is undetectable by current methodologies.

### SUMMARY

The invention features methods for identifying prostate cancer, or risk of recurrence of prostate cancer, by detecting nucleic acid methylation of one or more genes in one or more samples, and in particular in prostate tissue, blood and serum.

In a first aspect, the invention features a method for identifying a risk of developing prostate cancer in a subject comprising: detecting nucleic acid methylation of one or more genes in one or more samples, wherein detecting nucleic acid methylation identifies a risk of developing prostate cancer.

In one embodiment, the sample is one or more of blood, blood plasma, serum, cells, a cellular extract, a cellular aspirate, tissues, a tissue sample, or a tissue biopsy. In a related embodiment, the sample is taken from the prostate.

In a further embodiment, the one or more genes comprise one or more CpG islands. In another further embodiment, the one or more genes is selected from ASC (Apoptosis-associated speck-like protein containing a CARD) or CDH13 (Cadherin 13).

In another embodiment, methylation of at least one of the genes is detected. In another embodiment, methylation of at least two of the genes is detected. In a particular embodiment, at least one of the genes is ASC. In another embodiment, at least one of the genes is CDH13. In a further embodiment, at least two of the genes are ASC and CDH13.

In another aspect, the invention features a method for identifying a risk of developing prostate cancer in a subject comprising detecting nucleic acid methylation of at least one or more genes in a sample, wherein the genes are selected from ASC or CDH13, and wherein detecting nucleic acid methylation identifies a risk of developing prostate cancer.

In one embodiment, at least one of the genes is ASC. In another embodiment, at least one of the genes is CDH13. In another related embodiment, methylation of at least two genes is detected. In a related embodiment, the at least two genes are ASC and CDH13.

In another embodiment, methylation of ASC or CDH 13 indicates a risk for developing prostate cancer.

In one embodiment of any one of the above methods, the methylation of ASC and CDH13 indicates a higher risk for developing prostate cancer than methylation of ASC or CDH13 alone.

In another embodiment of any one of the above methods, the subject has previously been diagnosed with prostate cancer. In a related embodiment, the subject has previously been treated for prostate cancer.

In another embodiment of any one of the above methods, the detection is performed after surgery or therapy to treat a prostate cancer.

In another embodiment of any one of the above methods, the detection is used to predict the recurrence of prostate cancer.

In another embodiment of any one of the above methods, the detection is used to classify a subject as a low or high risk for prostate cancer recurrence.

In another embodiment of any one of the above methods, the detection used to determine a course of treatment for a subject.

In another aspect, the invention features a method for detecting or diagnosing prostate cancer in a subject comprising detecting nucleic acid methylation of one or more genes in one or more samples, wherein detecting nucleic acid methylation is used to detect or diagnose prostate cancer.

In still another aspect, the invention features a method for predicting the recurrence of prostate cancer in a subject comprising detecting nucleic acid methylation of one or more genes wherein detecting nucleic acid methylation of one or more genes is a predictor of the recurrence of prostate cancer.

In one embodiment of the above methods, the sample is one or more of blood, blood plasma, serum, cells, a cellular extract, a cellular aspirate, tissues, a tissue sample, or a tissue biopsy. In a related embodiment, the sample is taken from the prostate.

In one particular embodiment, the methylation of ASC or CDH13 is predictive of aggressive disease recurrence. In another embodiment, the methylation of ASC and CDH 13 indicates a higher risk for developing prostate cancer than methylation of ASC or CDH13 alone.

In another aspect, the invention features a method for determining the prognosis of a subject suffering from prostate cancer comprising detecting nucleic acid methylation of one or more genes wherein the detection of nucleic acid methylation is used for determining the prognosis of a subject suffering from prostate cancer.

In one embodiment, the prognosis determines course of treatment.

In one embodiment of any one of the above aspects, the subject is a human.

In another embodiment of any one of the above aspects, the method is performed prior to therapeutic intervention for prostate cancer.

In another further embodiment of any one of the above aspects, the method is performed after therapeutic intervention for prostate cancer. In a related embodiment, the therapeutic intervention is selected from treatment with an agent or surgery. In another related embodiment, the therapeutic intervention comprises radiation treatment.

In another further embodiment of any one of the above aspects, methylation is detected in CpG islands of the one or more genes.

In another aspect, the invention features a method for detecting or diagnosing prostate cancer in a subject comprising extracting nucleic acid from one or more cell or tissue samples and detecting nucleic acid methylation of one or more genes in the sample and identifying the nucleic acid methylation state of one or more genes, wherein nucleic acid methylation of genes indicates prostate cancer.

In another aspect, the invention features a method for predicting the recurrence of prostate cancer in a subject comprising extracting nucleic acid from one or more cell or tissue samples and detecting nucleic acid methylation of one or more genes in the sample; and identifying the nucleic acid methylation state of one or more genes, wherein nucleic acid methylation of genes is indicative of the recurrence of prostate cancer.

In one embodiment, the sample is one or more of blood, blood plasma, serum, cells, a cellular extract, a cellular aspirate, tissues, a tissue sample, or a tissue biopsy. In a related embodiment, the sample is taken from the prostate.

In one embodiment, the method determines the course of prostate cancer treatment.

In a further embodiment, the method is performed prior to therapeutic intervention for prostate cancer. In another embodiment, the method is performed after therapeutic intervention for prostate cancer. In a related embodiment, the therapeutic intervention is selected from treatment with an agent or surgery.

In another aspect, the invention features a method of treating a subject having or at risk for having prostate cancer comprising identifying nucleic acid methylation of one or more genes, where nucleic acid methylation indicates having or a risk for having prostate cancer and administering to the subject a therapeutically effective amount of a demethylating agent, thereby treating a subject having or at risk for having prostate cancer.

In one embodiment, the method is used in combination with one or more chemotherapeutic agents.

In one embodiment of any one of the above aspects, the method further comprises correlating the nucleic acid methylation of one or more genes in the sample to a methylation status of the gene.

In another particular embodiment, the methylation status is compared to a threshold value that distinguishes between individuals with and without prostate cancer.

In another embodiment of any one of the above aspects, the method further comprises comparing the nucleic acid methylation of one or more genes in the sample with a comparable samples obtained from a normal subject.

In one embodiment, detecting the nucleic acid methylation of one or more genes indicates the presence of prostate cancer.

In another embodiment, the methylation of at least one gene is detected. In a further embodiment, the methylation of at least two genes is detected. In a related embodiment, the genes are selected from ASC or CDH13. In another related embodiment, at least one of the genes is ASC. In still another related embodiment, at least one of the genes is CDH13. In a further embodiment, at least two of the genes are ASC and CDH13.

In one embodiment of any one of the above aspects, the detection of nucleic acid methylation is by a quantitative method.

In another embodiment of any one of the above aspects, the detection of nucleic acid methylation is carried out by polymerase chain reaction (PCR) analysis.

In one embodiment of any one of the above aspects, the detection of nucleic acid methylation is carried out by a method selected from: bisulfite sequencing, restriction endonuclease treatment and Southern blot analysis. In a further embodiment, the PCR is methylation specific PCR (MSP). In a related embodiment, the methylation specific PCR is multiplex methylation specific PCR.

In another embodiment of any one of the above aspects, the method of detecting nucleic acid methylation is performed as a high-throughput method.

In another embodiment of any one of the above aspects, the method is used in combination with the detection of other epigenetic markers. In a related embodiment, the other epigenetic markers are plasma or tumor epigenetic markers. In another related embodiment, the epigenetic marker is GST.

In another embodiment of any one of the above aspects, methylation is detected in CpG islands of the one or more genes. In a further embodiment, methylation is detected in CpG islands of the promoter region.

In another aspect, the invention features a kit for identifying the nucleic acid methylation state of one or more genes comprising gene specific primers for use in polymerase chain reaction (PCR), and instructions for use.

In still another aspect, the invention features a kit for detecting prostate cancer by detecting nucleic acid methylation of one or more genes, the kit comprising gene specific primers for use in polymerase chain reaction (PCR), and instructions for use.

In one embodiment of any of the above aspects, the PCR is methylation specific PCR (MSP). In another embodiment of the above-mentioned aspects, the one or more genes are ASC or CDH13.

In another embodiment of the above-mentioned aspects, the one or more genes comprise one or more CpG islands. In a related embodiment, the CpG islands are in the promoter region.

In another embodiment of any of the above aspects, the methylation of at least one of the genes is detected. In another embodiment of any of the above aspects, the methylation of at least two of the genes is detected. In a related embodiment, at least two of the genes are ASC and CHD13.

Other aspects of the invention are described infra.

### BRIEF DESCRIPTION OF THE FIGURES

FIGS. 1(A and B) shows the results of Methylation Specific Polymerase Chain reaction for ASC (Apoptosis-associated speck-like protein containing a CARD) (A) and GSTP-1 (glutathione S-transferase-1) (B).

FIG. 2 is a Table showing the demographic, clinical and pathologic characteristics for the 151 patients used in the study.

FIGS. 3(A and B) shows the nucleotide and corresponding amino acid sequences for (A) ASC (SEQ ID NO: 1 and 3) and (B) CDH13 (SEQ ID NO: 2 and 4).

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

The phrase “in combination with” is intended to refer to all forms of administration that provide a de-methylating agent, or the methods of the instant invention (e.g. methods of detection of methylation) together with a second agent, such as a chemotherapeutic agent, or a de-methylating agent, where the two are administered concurrently or sequentially in any order.

By “Apoptosis-associated speck-like protein containing a CARD” or ASC in certain embodiments is meant to refer to the protein encoded by NCBI accession No. NP_{—}037390 comprising SEQ ID NO: 1.

By “cadherin 13” (CDH13) in certain exemplary embodiments is meant to refer to the protein encoded by NCBI accession No. NP_{—}001248 comprising SEQ ID NO: 2.

The term “agent” as used herein is meant to refer to a polypeptide, polynucleotide, or fragment, or analog thereof, small molecule, or other biologically active molecule.

The term “CpG island” refers to a sequence of nucleic acid with an increased density relative to other nucleic acid regions of the dinucleotide CpG.

The term “epigenetic marker” or “epigenetic change” as used herein is meant to refer to a change in the DNA sequences or gene expression by a process or processes that do not change the DNA coding sequence itself. In an exemplary embodiment, methylation is an epigenetic marker.

As used herein, “methylation” is meant to refer to cytosine methylation at positions C5 or N4 of cytosine, the N6 position of adenine or other types of nucleic acid methylation. Methylation can be detection by, for example, by polymerase chain reaction (PCR), including, but not limited to methylation specific PCR. Portions of the DNA regions described herein will comprise at least one potential methylation site (i.e., a cytosine) and can comprise 2, 3, 4, 5, 6, 7, 8, 9, 10, or more potential methylation sites. In preferred embodiments, methylation is detected using methylation specific polymerase chain reaction (MSP).

As used herein the terms “methylation status” are meant to refer to the presence, absence and/or quantity of methylation at a particular nucleotide, or nucleotides within a portion of DNA. The methylation status of a particular DNA sequence (e.g., a DNA marker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.” A methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of methylation status. This is of particular use when it is desirable to compare the methylation status of a sequence in a sample to a threshold value. In certain examples, the methylation status is determined for a particular gene, for example a gene selected from ASC or CDH13. In preferred embodiments, methylation is detected using methylation specific polymerase chain reaction (MSP).

The phrase “nucleic acid” as used herein refers to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

The term “promoter” or “promoter region” refers to a minimal sequence sufficient to direct transcription or to render promoter-dependent gene expression that is controllable for cell-type specific, tissue-specific, or is inducible by external signals or agents. Promoters may be located in the 5′ or 3′ regions of the gene. Promoter regions, in whole or in part, of a number of nucleic acids can be examined for sites of CpG-island methylation.

The term “sample” as used herein refers to any biological or chemical mixture for use in the method of the invention. The sample can be a biological sample. The biological samples are generally derived from a patient, preferably as a bodily fluid (such as tumor tissue, lymph node, sputum, blood, bone marrow, cerebrospinal fluid, phlegm, saliva, or urine) or cell lysate. The cell lysate can be prepared from a tissue sample (e.g. a tissue sample obtained by biopsy), for example, a tissue sample (e.g. a tissue sample obtained by biopsy), blood, cerebrospinal fluid, phlegm, saliva, urine, or the sample can be cell lysate. In preferred examples, the sample is one or more of blood, blood plasma, serum, cells, a cellular extract, a cellular aspirate, tissues, a tissue sample, or a tissue biopsy. In preferred embodiments, the sample is from prostate cells, tissue or origin.

The term “stage” or “staging” as used herein is meant to refer to the extent or progression of proliferative disease, e.g. cancer, in a subject. Staging can be “clinical” and is according to the “stage classification” corresponding to the TNM classification (“Rinsho, Byori, Genpatsusei Kangan Toriatsukaikiyaku (Clinical and Pathological Codes for Handling Primary Liver Cancer)”: 22 p. Nihon Kangangaku Kenkyukai (Liver Cancer Study Group of Japan) edition (3rd revised edition), Kanehara Shuppan, 1992).

The term “subject” as used herein is meant to include vertebrates, preferably a mammal. Mammals include, but are not limited to, humans.

By “prostate cancer” is meant a cancer that forms in tissues of the prostate, a gland in the male reproductive system found below the bladder and in front of the rectum.

### DETAILED DESCRIPTION OF THE INVENTION

The invention is based upon the discovery that the methylation of certain genes can serve as prognostic and diagnostic markers for prostate cancer, and in particular for recurrent prostate cancer. DNA methylation of promoter regions leads to gene silencing in many cancers, and here, the impact of DNA methylation on the identification of recurrent prostate cancer is assessed. In particular, a methylation profile containing ASC and CDH13 is independently associated with an increased risk of biochemical recurrence in patients.

### I. Detection of Methylation

DNA methylases transfer methyl groups from the universal methyl donor S-adenosyl methionine to specific sites on the DNA. Several biological functions have been attributed to the methylated bases in DNA. The most established biological function for methylated DNA is the protection of DNA from digestion by cognate restriction enzymes. The restriction modification phenomenon has, so far, been observed only in bacteria. Mammalian cells, however, possess a different methylase that exclusively methylates cytosine residues that are 5′ neighbors of guanine (CpG). This modification of cytosine residues has important regulatory effects on gene expression, especially when involving CpG rich areas, known as CpG islands, located in the promoter regions of many genes.

Methylation has been shown by several lines of evidence to play a role in gene activity, cell differentiation, tumorigenesis, X-chromosome inactivation, genomic imprinting and other major biological processes (Razin, A., H., and Riggs, R. D. eds. in DNA Methylation Biochemistry and Biological Significance, Springer-Verlag, New York, 1984). In eukaryotic cells, methylation of cytosine residues that are immediately 5′ to a guanosine, occurs predominantly in CG poor regions (Bird, A., Nature, 321:209, 1986). In contrast, CpG islands remain unmethylated in normal cells, except during X-chromosome inactivation and parental specific imprinting (Li, et al., Nature, 366:362, 1993) where methylation of 5′ regulatory regions can lead to transcriptional repression. De novo methylation of the Rb gene has been demonstrated in a small fraction of retinoblastomas (Sakai, et al., Am. J. Hum. Genet., 48:880, 1991), and recently, a more detailed analysis of the VHL gene showed aberrant methylation in a subset of sporadic renal cell carcinomas (Herman, et al., Proc. Natl. Acad. Sci., U.S.A., 91:9700, 1994). Expression of a tumor suppressor gene can also be abolished by de novo DNA methylation of a normally unmethylated CpG island (Isla, et al., Nature Genet., 7:536, 1994; Herman, et al., supra; Merlo, et al., Nature Med., 1:686, 1995; Herman, et al., Cancer Res., 56:722, 1996; Graff, et al., Cancer Res., 55:5195, 1995; Herman, et al., Cancer Res., 55:4525, 1995).

In higher order eukaryotes DNA is methylated only at cytosines located 5′ to guanosine in the CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females. Aberrant methylation of normally unmethylated CpG islands has been described as a frequent event in immortalized and transformed cells, and has been associated with transcriptional inactivation of defined tumor suppressor genes in human cancers.

Any method that is sufficient to detect methylation is a suitable for use in the methods of the invention.

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa I, and Not I. Alternatively, chemical reagents can be used which selectively modify either the methylated or non-methylated form of CpG dinucleotide motifs.

Modified products can be detected directly, or after a further reaction which creates products which are easily distinguishable. Means which detect altered size and/or charge can be used to detect modified products, including but not limited to electrophoresis, chromatography, and mass spectrometry. Other means which are reliant on specific sequences can be used, including but not limited to hybridization, amplification, sequencing, and ligase chain reaction, Combinations of such techniques can be uses as is desired. Examples of such chemical reagents for selective modification include hydrazine and bisulfite ions. Hydrazine-modified DNA can be treated with piperidine to cleave it. Bisulfite ion-treated DNA can be treated with alkali.

Other techniques which can be used include technologies suitable for detecting DNA methylation with the use of bisulfite treatment include MSP, Mass Array, MethylLight, QAMA (quantitative analysis of methylated alleles), ERMA (enzymatic regional methylation assay), HeavyMethyl, pyrosequencing technology, MS-SNuPE, Methylquant, oligonucleotide-based microarray.

The ability to monitor the real-time progress of the PCR changes the way one approaches PCR-based quantification of DNA and RNA. Reactions are characterized by the point in time during cycling when amplification of a PCR product is first detected rather than the amount of PCR product accumulated after a fixed number of cycles. The higher the starting copy number of the nucleic acid target, the sooner a significant increase in fluorescence is observed. An amplification plot is the plot of fluorescence signal versus cycle number. In the initial cycles of PCR, there is little change in fluorescence signal. This defines the baseline for the amplification plot. An increase in fluorescence above the baseline indicates the detection of accumulated PCR product. A fixed fluorescence threshold can be set above the baseline. The parameter C_{T} (threshold cycle) is defined as the fractional cycle number at which the fluorescence passes the fixed threshold. For example, the PCR cycle number at which fluorescence reaches a threshold value of 10 times the standard deviation of baseline emission may be used as C_{T} and it is inversely proportional to the starting amount of target cDNA. A plot of the log of initial target copy number for a set of standards versus C_{T} is a straight line. Quantification of the amount of target in unknown samples is accomplished by measuring C_{T} and using the standard curve to determine starting copy number.

The entire process of calculating C_{TS}, preparing a standard curve, and determining starting copy number for unknowns can be performed by software, for example that of the 7700 system or 7900 system of Applied Biosystems. Real-time PCR requires an instrumentation platform that consists of a thermal cycler, computer, optics for fluorescence excitation and emission collection, and data acquisition and analysis software. These machines, available from several manufacturers, differ in sample capacity (some are 96-well standard format, others process fewer samples or require specialized glass capillary tubes), method of excitation (some use lasers, others broad spectrum light sources with tunable filters), and overall sensitivity. There are also platform-specific differences in how the software processes data. Real-time PCR machines are available at core facilities or labs that have the need for high throughput quantitative analysis.

Briefly, in the Q-PCR method the number of target gene copies can be extrapolated from a standard curve equation using the absolute quantitation method. For each gene, cDNA from a positive control is first generated from RNA by the reverse transcription reaction. Using about 1 μl of this cDNA, the gene under investigation is amplified using the primers by means of a standard PCR reaction. The amount of amplicon obtained is then quantified by spectrophotometry and the number of copies calculated on the basis of the molecular weight of each individual gene amplicon. Serial dilutions of this amplicon are tested with the Q-PCR assay to generate the gene specific standard curve. Optimal standard curves are based on PCR amplification efficiency from 90 to 100% (100% meaning that the amount of template is doubled after each cycle), as demonstrated by the slope of the standard curve equation. Linear regression analysis of all standard curves should show a high correlation (R² coefficient .gtoreq.0.98). Genomic DNA can be similarly quantified.

When measuring transcripts of a target gene, the starting material, transcripts of a housekeeping gene are quantified as an endogenous control. Beta-actin is one of the most used nonspecific housekeeping genes. For each experimental sample, the value of both the target and the housekeeping gene are extrapolated from the respective standard curve. The target value is then divided by the endogenous reference value to obtain a normalized target value independent of the amount of starting material.

The above-described quantitative real-time PCR methodology has been adapted to perform quantitative methylation-specific PCR (QM-MSP) by utilizing the external primers pairs in round one (multiplex) PCR and internal primer pairs in round two (real time MSP) PCR. Thus each set of genes has one pair of external primers and two sets of three internal primers/probe (internal sets are specific for unmethylated or methylated DNA). The external primer pairs can co-amplify a cocktail of genes, each pair selectively hybridizing to a member of the panel of genes being investigated using the invention method. The method of methylation-specific PCR (QM-MSP) has been described in US Patent Application 20050239101, incorporated by reference in its entirety herein.

methylation can be detected using two-stage, or “nested” PCR, for example as described in U.S. Pat. No. 7,214,485, incorporated by reference in its entirety herein. For example, two-stage, or “nested” polymerase chain reaction method is disclosed for detecting methylated DNA sequences at sufficiently high levels of sensitivity to permit cancer screening in biological fluid samples obtained non-invasively.

A method for assessment of the methylation status of any group of CpG sites within a CpG island, independent of the use of methylation-sensitive restriction enzymes, is described in U.S. Pat. No. 6,017,704 incorporated by reference in its entirety herein and described briefly as follows. This method employs primers that specific for the bisulfite reaction such that the PCR reaction itself is used to distinguish between the chemically modified methylated and unmethylated DNA, which adds an improved sensitivity of methylation detection. Unlike previous genomic sequencing methods for methylation identification which utilizes amplification primers which are specifically designed to avoid the CpG sequences, MSP primers themselves are specifically designed to recognize CpG sites to take advantage of the differences in methylation to amplify specific products to be identified by the invention assay. The methods of MSP include modification of DNA by sodium bisulfite or a comparable agent that converts all unmethylated but not methylated cytosines to uracil, and subsequent amplification with primers specific for methylated versus unmethylated DNA. This method of “methylation specific PCR” or MSP, requires only small amounts of DNA, is sensitive to 0.1% of methylated alleles of a given CpG island locus, and can be performed on DNA extracted from paraffin-embedded samples, for example. In addition, MSP eliminates the false positive results inherent to previous PCR-based approaches which relied on differential restriction enzyme cleavage to distinguish methylated from unmethylated DNA.

MSP provides significant advantages over previous PCR and other methods used for assaying methylation. MSP is markedly more sensitive than Southern analyses, facilitating detection of low numbers of methylated alleles and the study of DNA from small samples. MSP allows the study of paraffin-embedded materials, which could not previously be analyzed by Southern analysis. MSP also allows examination of all CpG sites, not just those within sequences recognized by methylation-sensitive restriction enzymes. This markedly increases the number of such sites which can be assessed and will allow rapid, fine mapping of methylation patterns throughout CpG rich regions. MSP also eliminates the frequent false positive results due to partial digestion of methylation-sensitive enzymes inherent in previous PCR methods for detecting methylation. Furthermore, with MSP, simultaneous detection of unmethylated and methylated products in a single sample confirms the integrity of DNA as a template for PCR and allows a semi-quantitative assessment of allele types which correlates with results of Southern analysis. Finally, the ability to validate the amplified product by differential restriction patterns is an additional advantage.

MSP can provide similar information as genomic sequencing, but can be performed with some advantages as follows. MSP is simpler and requires less time than genomic sequencing, with a typical PCR and gel analysis taking 4-6 hours. In contrast, genomic sequencing, amplification, cloning, and subsequent sequencing may take days. MSP also avoids the use of expensive sequencing reagents and the use of radioactivity. Both of these factors make MSP better suited for the analysis of large numbers of samples. The use of PCR as the step to distinguish methylated from unmethylated DNA in MSP allows for significant increase in the sensitivity of methylation detection. For example, if cloning is not used prior to genomic sequencing of the DNA, less than 10% methylated DNA in a background of unmethylated DNA cannot be seen (Myohanen, et al., supra). The use of PCR and cloning does allow sensitive detection of methylation patterns in very small amounts of DNA by genomic sequencing (Frommer, et al., Proc. Natl. Acad. Sci. USA, 89:1827, 1992; Clark, et al., Nucleic Acids Research, 22:2990, 1994). However, this means in practice that it would require sequencing analysis of 10 clones to detect 10% methylation, 100 clones to detect 1% methylation, and to reach the level of sensitivity we have demonstrated with MSP (1:1000), one would have to sequence 1000 individual clones.

“Multiplex methylation-specific PCR” is a unique version of methylation-specific PCR. Methylation-specific PCR is described in U.S. Pat. Nos. 5,786,146; 6,200,756; 6,017,704 and 6,265,171, each of which is incorporated herein by reference in its entirety. Multiplex methylation-specific PCR utilizes MSP primers for a multiplicity of markers, for example three or more different markers, in a two-stage nested PCR amplification reaction. The primers used in the first PCR reaction are selected to amplify a larger portion of the target sequence than the primers of the second PCR reaction. The primers used in the first PCR reaction are referred to herein as “external primers” or DNA primers” and the primers used in the second PCR reaction are referred to herein as “MSP primers.” Two sets of primers (i.e., methylated and unmethylated for each of the markers targeted in the reaction) are used as the MSP primers. In addition in multiplex methylation-specific PCR, as described herein, a small amount (i.e., 1 μl) of a 1:10 to about 10⁶ dilution of the reaction product of the first “external” PCR reaction is used in the second “internal” MSP PCR reaction.

The term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded. If double stranded, the primer is first treated to separate its strands before being used to prepare extension products. Preferably, the primer is an oligodeoxy ribonucleotide. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it may contain fewer nucleotides.

Primers of the invention are designed to be “substantially” complementary to each strand of the oligonucleotide to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with a 5′ and 3′ oligonucleotide to hybridize therewith and permit amplification of CpG containing nucleic acid sequence.

Primers of the invention are employed in the amplification process, which is an enzymatic chain reaction that produces exponentially increasing quantities of target locus relative to the number of reaction steps involved (e.g., polymerase chain reaction or PCR). Typically, one primer is complementary to the negative (−) strand of the locus (antisense primer) and the other is complementary to the positive (+) strand (sense primer). Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

The oligonucleotide primers used in invention methods may be prepared using any suitable method, such as conventional phosphotriester and phosphodiester methods or automated embodiments thereof. In one such automated embodiment, diethylphos-phoramidites are used as starting materials and may be synthesized as described by Beaucage, et al. (Tetrahedron Letters, 22:1859-1862, 1981). One method for synthesizing oligonucleotides on a modified solid support is described in U.S. Pat. No. 4,458,066.

The primers used in the invention for amplification of the CpG-containing nucleic acid in the specimen, after bisulfite modification, specifically distinguish between untreated or unmodified DNA, methylated, and non-methylated DNA. MSP primers for the non-methylated DNA preferably have a T in the 3′ CG pair to distinguish it from the C retained in methylated DNA, and the complement is designed for the antisense primer. MSP primers usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention embrace oligonucleotides of sufficient length and appropriate sequence so as to provide specific initiation of polymerization on a significant number of nucleic acids in the polymorphic locus. Where the nucleic acid sequence of interest contains two strands, it is necessary to separate the strands of the nucleic acid before it can be used as a template for the amplification process. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80.degree. to 105.degree C. for times ranging from about 1 to 10 minutes. Strand separation may also be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Aim. Rev. Genetics, 16:405-437, 1982).

As described herein, any nucleic acid specimen, in purified or nonpurified form, can be utilized as the starting nucleic acid or acids, provided it contains, or is suspected of containing, the specific nucleic acid sequence containing the target locus (e.g., CpG).

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. Preferably, a molar excess (for genomic nucleic acid, usually about 10⁸:1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated lona-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90 C-100 C. from about 1 to 10 minutes, preferably from 1 to 4 minutes. After this heating period, the solution is allowed to cool to room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40 C. Most conveniently the reaction occurs at room temperature.

In certain preferred embodiments, the agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation). Suitable enzymes will facilitate combination of the nucleotides in the proper manner to form the primer extension products which are complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Preferably, the method of amplifying is by PCR, as described herein and as is commonly used by those of ordinary skill in the art. Alternative methods of amplification have been described and can also be employed as long as the methylated and non-methylated loci amplified by PCR using the primers of the invention is similarly amplified by the alternative means.

The amplified products are preferably identified as methylated or non-methylated by sequencing. Sequences amplified by the methods of the invention can be further evaluated, detected, cloned, sequenced, and the like, either in solution or after binding to a solid support, by any method usually applied to the detection of a specific DNA sequence such as PCR, oligomer restriction (39), allele-specific oligonucleotide (ASO) probe analysis (40), oligonucleotide ligation assays (OLAs) (41), and the like. Molecular techniques for DNA analysis have been reviewed (42).

Optionally, the methylation pattern of the nucleic acid can be confirmed by restriction enzyme digestion and Southern blot analysis. Examples of methylation sensitive restriction endonucleases which can be used to detect 5′CpG methylation include SmaI, SacII, EagI, MspI, HpaII, BstUI and BssHII, for example.

The invention provides a method for detecting a cell having a methylated CpG island or a cell proliferative disorder associated with methylated CpG in a tissue or biological fluid of a subject, comprising contacting a target cellular component suspected of expressing a gene having a methylated CpG or having a CpG-associated disorder, with an agent which binds to the component. The target cell component can be nucleic acid, such as DNA or RNA, or protein. When the component is nucleic acid, the reagent is a nucleic acid probe or PCR primer. When the cell component is protein, the reagent is an antibody probe. The probes can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the antibody, or will be able to ascertain such, using routine experimentation.

One may use MALDI mass spectrometry in combination with a methylation detection assay to observe the size of a nucleic acid product. The principle behind mass spectrometry is the ionizing of nucleic acids and separating them according to their mass to charge ratio. Similar to electrophoresis, one can use mass spectrometry to detect a specific nucleic acid that was created in an experiment to determine methylation. See Tost, J. et al. Analysis and accurate quantification of CpG methylation by MALDI mass spectrometry. Nuc Acid Res, 2003, 31, 9

One form of chromatography, high performance liquid chromatography, is used to separate components of a mixture based on a variety of chemical interactions between a substance being analyzed and a chromatography column. DNA is first treated with sodium bisulfite, which converts an unmethylated cytosine to uracil, while methylated cytosine residues remain unaffected. One may amplify the region containing potential methylation sites via PCR and separate the products via denaturing high performance liquid chromatography (DHPLC). DHPLC has the resolution capabilities to distinguish between methylated (containing cytosine) and unmethylated (containing uracil) DNA sequences. See Deng, D. et al. Simultaneous detection of CpG methylation and single nucleotide polymorphism by denaturing high performance liquid chromatography. 2002 Nuc Acid Res, 30, 3.

Hybridization is a technique for detecting specific nucleic acid sequences that is based on the annealing of two complementary nucleic acid strands to form a double-stranded molecule. In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2.times.SSC/0.1% SDS at about room temperature (hybridization conditions); 0.2.times.SSC/0.1% SDS at about room temperature (low stringency conditions); 0.2.times.SSC/0.1% SDS at about 42.degree. C. (moderate stringency conditions); and 0.1.times.SSC at about 68.degree. C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

One example of the use of hybridization is a microarray assay to determine the methylation status of DNA. After sodium bisulfite treatment of DNA, which converts an unmethylated cytosine to uracil while methylated cytosine residues remain unaffected, oligonucleotides complementary to potential methylation sites can hybridize to the bisulfite-treated DNA. The oligonucleotides are designed to be complimentary to either sequence containing uracil or sequence containing cytosine, representing unmethylated and methylated DNA, respectively. Computer-based microarray technology can determine which oligonucleotides hybridize with the DNA sequence and one can deduce the methylation status of the DNA.

An additional method of determining the results after sodium bisulfite treatment would be to sequence the DNA to directly observe any bisulfite-modifications. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a pyrogram. Pyrosequencing can exploit the sequence differences that arise following sodium bisulfite-conversion of DNA.

A variety of amplification techniques may be used in a reaction for creating distinguishable products. Some of these techniques employ PCR. Other suitable amplification methods include the ligase chain reaction (LCR) (Barringer et al, 1990), transcription amplification (Kwoh et al. 1989; WO88/10315), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995), nucleic acid based sequence amplification (NASBA) (U.S. Pat. Nos. 5,409,818; 5,554,517; 6,063,603), nick displacement amplification (WO2004/067726).

Sequence variation that reflects the methylation status at CpG dinucleotides in the original genomic DNA offers two approaches to PCR primer design. In the first approach, the primers do not themselves “cover” or hybridize to any potential sites of DNA methylation; sequence variation at sites of differential methylation are located between the two primers. Such primers are used in bisulphite genomic sequencing, COBRA, Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues.

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfate ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

Another way to distinguish between modified and nonmodified DNA is to use oligonucleotide probes which may also be specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Still another way for the identification of methylated CpG dinucleotides utilizes the ability of the MBD domain of the McCP2 protein to selectively bind to methylated DNA sequences (Cross et al, 1994; Shiraishi et al, 1999). Restriction enconuclease digested genomic DNA is loaded onto expressed His-tagged methyl-CpG binding domain that is immobilized to a solid matrix and used for preparative column chromatography to isolate highly methylated DNA sequences.

Real time chemistry allows for the detection of PCR amplification during the early phases of the reactions, and makes quantitation of DNA and RNA easier and more precise. A few variations of the real-time PCR are known. They include the TaqMan.™. system and Molecular Beacon.™. system which have separate probes labeled with a fluorophore and a fuorescence quencher. In the Scorpion.™. system the labeled probe in the form of a hairpin structure is linked to the primer.

DNA methylation analysis has been performed successfully with a number of techniques which include the MALDI-TOFF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, and Oligonucleotide-based microarray systems.

The number of genes whose silencing is tested and/or detected can vary: one, two, three, four, five, or more genes can be tested and/or detected. In some examples, methylation of at least one gene is detected. In other examples, methylation of at least two genes is detected. However, methylation of any number of genes may be detected, using the methods as described herein.

For purposes of the invention, an antibody or nucleic acid probe specific for a gene or gene product may be used to detect the presence of methylation either by detecting the level of polypeptide (using antibody) or methylation of the polynucleotide (using nucleic acid probe) in biological fluids or tissues. For antibody-based detection, the level of the polypeptide is compared with the level of polypeptide found in a corresponding “normal” tissue.

Oligonucleotide primers based on any coding sequence region of the promoter in gene selected from genes involved in tumor suppression, nucleic acid repair, apoptosis, anti-proliferation, ras signaling, adhesion, differentiation, development, and cell cycle regulation. In particular, oligonucleotide primers are based on the coding sequence region of the promoter in a gene selected from, for example ASC or CDH13, and are useful for amplifying DNA, for example by PCR.

ASC was first identified in a study as a 22-kDa protein that exhibited aggreagation and appeared as a speck during apoptosis induced by retinoic acid and other anti-tumor drugs (Masumoto et al. J Biol Chem, Vol. 274, Issue 48, 33835-33838, Nov. 26, 1999, incorporated by reference in its entirety herein). Cloning and sequencing of its cDNA revealed that the protein comprised 195 amino acids and that its C-terminal half has a caspase recruitment domain (CARD) motif, characteristic of numerous proteins involved in apoptotic signaling. The identified protein was referred to as ASC (apoptosis-associated speck-like protein containing a CARD). The ASC gene was mapped on chromosome 16p11.2-12.

ASC, in certain exemplary embodiments, corresponds to the nucleotide sequence represented by NCBI accession No. NM_{—}145182 (SEQ ID NO: 3), shown below, and the corresponding amino acid sequence encoded by NCBI accession No. NP_{—}037390 comprising SEQ ID NO: 1, also shown below:

| SEQ ID NO: 3 | |
|---|---|
| 1 | gagggcgcga tcctggcgtc ccccgacggc ctggggcccc aatccagagg cctgggtggg |
| 61 | aggggaccaa gggtgtagta aggaagcgcc ttttgctgga gggcaacgga ccggggcggg |
| 121 | gagtcgggag accagagtgg gaggaaggcg gggagtccag gttccgcccc ggagccgact |
| 181 | tcctcctggt cggcggctgc agcggggtga gcggcggcag cggccgggga tcctggagcc |
| 241 | atggggcgcg cgcgcgacgc catcctggat gcgctggaga acctgaccgc cgaggagctc |
| 301 | aagaagttca agctgaagct gctgtcggtg ccgctgcgcg agggctacgg gcgcatcccg |
| 361 | cggggcgcgc tgctgtccat ggacgccttg gacctcaccg acaagctggt cagcttctac |
| 421 | ctggagacct acggcgccga gctcaccgct aacgtgctgc gcgacatggg cctgcaggag |
| 481 | atggccgggc agctgcaggc ggccacgcac cagggcctgc actttataga ccagcaccgg |
| 541 | gctgcgctta tcgcgagggt cacaaacgtt gagtggctgc tggatgctct gtacgggaag |
| 601 | gtcctgacgg atgagcagta ccaggcagtg cgggccgagc ccaccaaccc aagcaagatg |
| 661 | cggaagctct tcagtttcac accagcctgg aactggacct gcaaggactt gctcctccag |
| 721 | gccctaaggg agtcccagtc ctacctggtg gaggacctgg agcggagctg aggctccttc |
| 781 | ccagcaacac tccggtcagc ccctggcaat cccaccaaat catcctgaat ctgatctttt |
| 841 | tatacacaat atacgaaaag ccagcttgaa aaaaaaaaa |
| SEQ ID NO: 1 | |
| 1 | mgrardaild alenltaeel kkfklkllsv plregygrip rgallsmdal dltdklvsfy |
| 61 | letygaelta nvlrdmglqe magqlqaath qgsgaapagi qappqsaakp glhfidqhra |
| 121 | aliarvtnve wlldalygkv ltdeqyqavr aeptnpskmr klfsftpawn wtckdlllqa |
| 181 | lresqsylve dlers |

CDH13 (cadherin 13) is a member of the cadherin superfamily. The encoded protein is a calcium dependent cell-cell adhesion glycoprotein comprised of five extracellular cadherin repeats, a transmembrane region but, unlike the typical cadherin superfamily member, lacks the highly conserved cytoplasmic region.

CDH13, in certain exemplary embodiments, corresponds to the nucleotide sequence represented by NCBI accession No. NM_{—}001257 (SEQ ID NO: 4), shown below, and the corresponding amino acid sequence encoded by NCBI accession No. NP_{—}001248 comprising SEQ ID NO: 2, also shown below:

| SEQ ID NO: 4 | |
|---|---|
| 1 | gggaagttgg ctggctggcg aggcagagcc tctcctcaaa gcctggctcc cacggaaaat |
| 61 | atgctcagtg cagccgcgtg catgaatgaa aacgccgccg ggcgcttcta gtcggacaaa |
| 121 | atgcagccga gaactccgct cgttctgtgc gttctcctgt cccaggtgct gctgctaaca |
| 181 | tctgcagaag atttggactg cactcctgga tttcagcaga aagtgttcca tatcaatcag |
| 241 | ccagctgaat tcattgagga ccagtcaatt ctaaacttga ccttcagtga ctgtaaggga |
| 301 | aacgacaagc tacgctatga ggtctcgagc ccatacttca aggtgaacag cgatggcggc |
| 361 | ttagttgctc tgagaaacat aactgcagtg ggcaaaactc tgttcgtcca tgcacggacc |
| 421 | ccccatgcgg aagatatggc agaactcgtg attgtcgggg ggaaagacat ccagggctcc |
| 481 | ttgcaggata tatttaaatt tgcaagaact tctcctgtcc caagacaaaa gaggtccatt |
| 541 | gtggtatctc ccattttaat tccagagaat cagagacagc ctttcccaag agatgttggc |
| 601 | aaggtagtcg atagtgacag gccagaaagg tccaagttcc ggctcactgg aaagggagtg |
| 661 | gatcaagagc ctaaaggaat tttcagaatc aatgagaaca cagggagcgt ctccgtgaca |
| 721 | cggaccttgg acagagaagt aatcgctgtt tatcaactat ttgtggagac cactgatgtc |
| 781 | aatggcaaaa ctctcgaggg gccggtgcct ctggaagtca ttgtgattga tcagaatgac |
| 841 | aaccgaccga tctttcggga aggcccctac atcggccacg tcatggaagg gtcacccaca |
| 901 | ggcaccacag tgatgcggat gacagccttt gatgcagatg acccagccac cgataatgcc |
| 961 | ctcctgcggt ataatatccg tcagcagacg cctgacaagc catctcccaa catgttctac |
| 1021 | atcgatcctg agaaaggaga cattgtcact gttgtgtcac ctgcgctgct ggaccgagag |
| 1081 | actctggaaa atcccaagta tgaactgatc atcgaggctc aagatatggc tggactggat |
| 1141 | gttggattaa caggcacggc cacagccacg atcatgatcg atgacaaaaa tgatcactca |
| 1201 | ccaaaattca ccaagaaaga gtttcaagcc acagtcgagg aaggagctgt gggagttatt |
| 1261 | gtcaatttga cagttgaaga taaggatgac cccaccacag gtgcatggag ggctgcctac |
| 1321 | accatcatca acggaaaccc cgggcagagc tttgaaatcc acaccaaccc tcaaaccaac |
| 1381 | gaagggatgc tttctgttgt caaaccattg gactatgaaa tttctgcctt ccacaccctg |
| 1441 | ctgatcaaag tggaaaatga agacccactc gtacccgacg tctcctacgg ccccagctcc |
| 1501 | acagccaccg tccacatcac tgtcctggat gtcaacgagg gcccagtctt ctacccagac |
| 1561 | cccatgatgg tgaccaggca ggaggacctc tctgtgggca gcgtgctgct gacagtgaat |
| 1621 | gccacggacc ccgactccct gcagcatcaa accatcaggt attctgttta caaggaccca |
| 1681 | gcaggttggc tgaatattaa ccccatcaat gggactgttg acaccacagc tgtgctggac |
| 1741 | cgtgagtccc catttgtcga caacagcgtg tacactgctc tcttcctggc aattgacagt |
| 1801 | ggcaaccctc ccgctacggg cactgggact ttgctgataa ccctggagga cgtgaatgac |
| 1861 | aatgccccgt tcatttaccc cacagtagct gaagtctgtg atgatgccaa aaacctcagt |
| 1921 | gtagtcattt tgggagcatc agataaggat cttcacccga atacagatcc tttcaaattt |
| 1981 | gaaatccaca aacaagctgt tcctgataaa gtctggaaga tctccaagat caacaataca |
| 2041 | cacgccctgg taagccttct tcaaaatctg aacaaagcaa actacaacct gcccatcatg |
| 2101 | gtgacagatt cagggaaacc acccatgacg aatatcacag atctcagggt acaagtgtgc |
| 2161 | tcctgcagga attccaaagt ggactgcaac gcggcagggg ccctgcgctt cagcctgccc |
| 2221 | tcagtcctgc tcctcagcct cttcagctta gcttgtctgt gagaactcct gacgtctgaa |
| 2281 | gcttgactcc caagtttcca tagcaacagg aaaaaaaaaa atctatccaa atctgaagat |
| 2341 | tgcggtttac agctatcgaa cttcacaact aggcctcaat tgttccggtt ttttattttc |
| 2401 | tttacaattt cacttagtct gtacttcatc attttgacag catcttcctc cctcctttaa |
| 2461 | ttaatggaat cttctgaatt ttccctgaat gtttaaagat catgacatat gacttgatct |
| 2521 | tctgggagca ggaacaatga ctactttttc tggtgtgtta acatgtcgct agccagtgct |
| 2581 | ccaggcaccc agctttgtct gtgggttagt attggtgtat gtatgagtat ctgtatgtat |
| 2641 | atatacacgg tatttataga gagagactat cctggagaag cctcgttttg atgccattct |
| 2701 | tccttgcaag gttaagcaag gtgggtggaa actaagacac ctgaaccctc cagggcctcc |
| 2761 | cgcatcaagg tcagcatgag gacagaccac agagctgtca cttttgctcc gaagctactt |
| 2821 | ctccactgtc ccgttcagtc tgaatgctgc cacaaccagc caggcaggtc cacagagagg |
| 2881 | gagagcagag aaagaagtcc tttctcttta ttgagttcga ggactacaac caatttacac |
| 2941 | tgccatctga tgccgtgatc ctgagccaag gaggtgagga gcagagcagg caatttcacc |
| 3001 | accaaatgcc aagaaaaggg ctgacatttt ctttcatggg caccaacctg catttgtatg |
| 3061 | tgtcccgaat ccacagtcgt actgattcta atggggacac agatcatggt agagaatctc |
| 3121 | tccctcctca gtaaatgtac aactgcacct gtcatcatgg aggtcataca tgcatacaaa |
| 3181 | gaggtgtaca ggtaccatct tgtatacaca tatataccca catgtacaga catacattta |
| 3241 | tgcacattca cgctgtttgt ttcatatata caggcataaa atagagtaaa tacaggtagt |
| 3301 | tttaaaagta cccttttgtg tgaattgact accgttgttt gcaaacccga aaataaaaga |
| 3361 | cgttcattat gtatgaaaag taactgattt gtattctgtg agcatgtaaa agcggaaagt |
| 3421 | tagtgcttgt tctaagatta ccttcttgtt gataaaccat aaatgaatca tcaaagctca |
| 3481 | caccaaattt ttctatcaaa taaaactagt gacagcttgt ggctttttat tagagctcgc |
| 3541 | cacgaactag ggtaaggtga gtgtcttagc atattttaat gcagttgctt actaaaggtt |
| 3601 | ttaaccgcac atgcacacac acacgctttc ttatgcaatc tatgtttgca cttgtgcttt |
| 3661 | cagttagcct tctgtaggaa gtagaagtca tatgttgtct ttgttgtagt gaaattatac |
| 3721 | agatagagtt ccatatattg tatttgtttc aatggtaaat ccttttggaa catatagaat |
| 3781 | gcagagattt ttttttccat taaaataaat gggtattggt ggttaaaaaa aaaaaaaaaa |
| 3841 | aa |
| SEQ ID NO: 2 | |
| | mqprtplvlc vllsqvlllt saedldctpg fqqkvfhinq paefiedqsi lnltfsdckg |
| 61 | ndklryevss pyfkvnsdgg lvalrnitav gktlfvhart phaedmaelv ivggkdiqgs |
| 121 | lqdifkfart spvprqkrsi vvspilipen qrqpfprdvg kvvdsdrper skfrltgkgv |
| 181 | dqepkgifri nentgsvsvt rtldreviav yqlfvettdv ngktlegpvp levividqnd |
| 241 | nrpifregpy ighvmegspt gttvmrmtaf daddpatdna llrynirqqt pdkpspnmfy |
| 301 | idpekgdivt vvspalldre tlenpkyeli ieaqdmagld vgltgtatat imiddkndhs |
| 361 | pkftkkefqa tveegavgvi vnltvedkdd pttgawraay tiingnpgqs feihtnpqtn |
| 421 | egmlsvvkpl dyeisafhtl likvenedpl vpdvsygpss tatvhitvld vnegpvfypd |
| 481 | pmmvtrqedl svgsvlltvn atdpdslqhq tirysvykdp agwlninpin gtvdttavld |
| 541 | respfvdnsv ytalflaids gnppatgtgt llitledvnd napfiyptva evcddaknls |
| 601 | vvilgasdkd lhpntdpfkf eihkqavpdk vwkiskinnt halvsllqnl nkanynlpim |
| 661 | vtdsgkppmt nitdlrvqvc scrnskvdcn aagalrfslp svlllslfsl acl |

These genes are merely listed as examples and are not meant to be limiting.

Any specimen containing a detectable amount of polynucleotide or antigen can be used. Preferably the subject is human.

The present invention assesses the impact of DNA methylation on the identification of recurrent prostate cancer, and in particular reports that a methylation profile containing ASC and CDH13 is independently associated with an increased risk of biochemical recurrence in patients who have undergone radical prostatectomies . These markers are shown to also be potential targets for reversal of gene silencing and may be important in adjuvant approaches to reduce disease recurrence.

Using the methods of the invention, expression of any gene, such as genes involved in tumor suppression, nucleic acid repair, apoptosis, anti-proliferation, ras signaling, adhesion, differentiation, development, and cell cycle regulation, can be identified in a cell and the appropriate course of treatment can be employed (e.g., sense gene therapy or drug therapy). The expression pattern of the gene may vary with the stage of malignancy of a cell, therefore, a sample can be screened with a panel of gene or gene product specific reagents (i.e., nucleic acid probes or antibodies) to detect gene expression and then diagnose the stage of malignancy of the cell.

Any of the methods as described herein can be used in high throughput analysis of DNA methylation. For example, U.S. Pat. No. 7,144,701, incorporated by reference in its entirety herein, describes differential methylation hybridization (DMH) for a high-throughput analysis of DNA methylation.

### II. Methods of Detection and Diagnosis

In mammals, conditions associated with aberrant methylation of genes that can be detected or monitored include, but are not limited to, metastases associated with carcinomas and sarcomas of all kinds, including one or more specific types of cancer, e.g., a lung cancer, breast cancer, an alimentary or gastrointestinal tract cancer such as colon, esophageal and pancreatic cancer, a liver cancer, a skin cancer, an ovarian cancer, an endometrial cancer, a prostate cancer, a lymphoma, hematopoietic tumors, such as a leukemia, a kidney cancer, a bronchial cancer, a muscle cancer, a bone cancer, a bladder cancer or a brain cancer, such as astrocytoma, anaplastic astrocytoma, glioblastoma, medulloblastoma, and neuroblastoma and their metastases. Suitable pre-malignant lesions to be detected or monitored using the invention include, but are not limited to, lobular carcinoma in situ and ductal carcinoma in situ.

The methods of the invention as described herein are used in certain exemplary embodiments to identify prostate cancer, or the risk of recurrence of prostate cancer, by detecting the methylation of one or more genes in one or more samples. In this way, the detection of nucleic acid methylation identifies or identifies a risk for recurrence of prostate cancer.

The invention methods can be used to assay the DNA of any mammalian subject, including, but not limited to, humans, pet (e.g., dogs, cats, ferrets) and farm animals (meat and dairy).

The invention features in certain aspects a method for identifying prostate cancer in a subject comprising detecting nucleic acid methylation of one or more genes in one or more samples, wherein detecting nucleic acid methylation identifies prostate cancer.

The samples, in certain embodiments, can be from one or more of blood, blood plasma, serum, cells, a cellular extract, a cellular aspirate, tissues, a tissue sample, or a tissue biopsy. Thus, the invention can be used to identify prostate cancer in a subject comprising detecting nucleic acid methylation of one or more genes in a sample, wherein detecting nucleic acid methylation identifies prostate cancer.

In other aspects, the invention features a method for identifying prostate cancer in a subject comprising detecting nucleic acid methylation of one or more genes in the sample, where the genes are selected from wherein the one or more genes is selected from ASC or CDH13. In certain cases, methylation of just one of the genes is detected, for example methylation of just ASC is detected. In other cases, methylation of just CDH13 is detected. In other examples, methylation of two or more genes is detected, for example methylation of both ASC and CDH13 is detected.

Thus, the invention features methods for identifying a risk of developing prostate cancer in a subject comprising detecting nucleic acid methylation of at least one or more genes in a sample, wherein the genes are selected from ASC or CDH13, and wherein detecting nucleic acid methylation identifies a risk of developing prostate cancer.

In certain cases, when methylation of two genes is detected, this is indicative of a higher risk of recurrence of prostate cancer than when methylation of one gene is detected. In other certain cases, when methylation of two genes is detected, this is indicative of a more aggressive form of prostate cancer than when methylation of one gene is detected. In certain cases, when a higher risk of recurrence or a more aggressive disease is detected, a clinician may recommend a course of treatment based on such detection. Patient prognosis may be determined based on the number of methylated genes detected, in certain examples.

In other examples, the invention as described herein features methods for detecting or diagnosing prostate cancer in a subject comprising detecting nucleic acid methylation of one or more genes in one or more samples, wherein detecting nucleic acid methylation is used to detect or diagnose prostate cancer.

In practice, the method for detecting or diagnosing prostate cancer in a subject method for detecting or diagnosing prostate cancer in a subject comprising extracting nucleic acid from one or more cell or tissue samples, detecting nucleic acid methylation of one or more genes in the sample, identifying the nucleic acid methylation state of one or more genes, wherein nucleic acid methylation of genes indicates prostate cancer.

As described herein, in certain preferred examples, the one or more genes comprise one or more CpG islands in the promoter regions. Accordingly, any gene that contains one or more CpG island in the promoter region is suitable for use in the methods of the invention; however in certain preferred examples, the one or more genes may be selected from any of the genes described in the application herein, for example the genes in Table 1. In preferred examples, the genes are selected from ASC and CDH13.

In certain embodiments, methylation of at least one of the genes is detected. In other certain embodiments, methylation of at least two of the genes is detected.

The detection of methylation as described in these methods can be used after surgery or therapy to treat a prostate cancer.

The detection of methylation as described in these methods can be used to predict the recurrence of a prostate cancer.

The detection of methylation as described in these methods can be used to stage or determine the progression of prostate cancer.

The detection of methylation as described in these methods can be used to determine a course of treatment for a subject.

These embodiments are discussed in further detail herein.

Methods of Treatment

The invention as described herein can be used to treat a subject having or at risk for having a prostate cancer. Accordingly, the method comprises identifying nucleic acid methylation of one or more genes, where nucleic acid methylation indicates having or a risk for having prostate cancer; and administering to the subject a therapeutically effective amount of a demethylating agent, thereby treating a subject having or at risk for having prostate cancer.

The method can be used in combination with one or more chemotherapeutic agents. Anti-cancer drugs that may be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol,aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfornithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

Demethylating Agents

In certain embodiments, the invention features methods of identifying an agent that de-methylates methylated nucleic acids comprising identifying one or more cell or tissue samples with methylated nucleic acid, extracting the methylated nucleic acid, contacting the nucleic acid with one or more nucleic acid de-methylating candidate agents and a control agent, and identifying the nucleic acid methylation state, wherein nucleic acid de-methylation of genes in the sample by the candidate agent compared to the control indicates a demethylating agent, thereby identifying an agent that de-methylates methylated nucleic acid.

Demethylating agents include, but are not limited to, 5-aza-2′-deoxycytidine, 5-aza-cytidine, Zebularine, procaine, and L-ethionine.

Another way to restore epigenetically silenced gene expression is to introduce a non-methylated polynucleotide into a cell, so that it will be expressed in the cell. Various gene therapy vectors and vehicles are known in the art and any can be used as is suitable for a particular situation. Certain vectors are suitable for short term expression and certain vectors are suitable for prolonged expression. Certain vectors are trophic for certain organs and these can be used as is appropriate in the particular situation. Vectors may be viral or non-viral. The polynucleotide can, but need not, be contained in a vector, for example, a viral vector, and can be formulated, for example, in a matrix such as a liposome, microbubbles. The polynucleotide can be introduced into a cell by administering the polynucleotide to the subject such that it contacts the cell and is taken up by the cell and the encoded polypeptide expressed. Preferably the specific polynucleotide will be one which the patient has been tested for and been found to carry a silenced version. The polynucleotides for treating prostate cancer will typically encode a gene selected from ASC or CDH13.

### III. Methods of Predicting Disease Recurrence

In other certain aspects, the invention features methods for predicting the recurrence of prostate cancer.

Accordingly, the invention features methods for predicting the recurrence of prostate cancer in a subject comprising detecting nucleic acid methylation of one or more genes wherein detecting nucleic acid methylation of one or more genes is a predictor of the recurrence of prostate cancer.

In certain preferred embodiments, the method comprises extracting nucleic acid from one or more cell or tissue samples, detecting nucleic acid methylation of one or more genes in the sample, and identifying the nucleic acid methylation state of one or more genes, wherein nucleic acid methylation of genes is indicative of the recurrence of prostate cancer.

In certain cases, the rate of recurrence of prostate cancer can be correlated with the detection of methylation in a cell or tissue sample. In certain embodiments, the cell or tissue sample is one or more of blood, blood plasma, serum, cells, a cellular extract, a cellular aspirate, tissues, a tissue sample, or a tissue biopsy. The sample is, in exemplary embodiments, derived from the prostate. In certain embodiments, the rate of recurrence of prostate cancer is more rapid when gene methylation is detected in two or more genes than one gene. For example, when gene methylation (e.g. ASC or CDH13) is detected in either of ASC or CDH13, the odds of recurrence may be less than when both genes are methylated.

The discovery and clinical validation of markers for cancer of all types which can predict prognosis, likelihood of invasive or metastatic spread is one of the major challenges facing in the field of oncology today. Adjuvant and neoadjuvant therapy (e.g. chemotherapy) are promising treatment modalities, however although adjuvant chemotherapy has been demonstrated to improve survival, for example in node negative breast cancer patients (43), problems remain, for example in the uncertainty as to how to best identify patients whose risk of disease recurrence exceeds their risk of significant therapeutic toxicity. Thus, a need remains for methods for that enable clinical decisions on adjuvant and neoadjuvant therapy, tumor surveillance and the likelihood of disease progression based on validated tumor markers.

In other certain aspects, the invention features a method for determining the prognosis of a subject suffering from prostate cancer comprising: detecting nucleic acid methylation of one or more genes wherein the detection of nucleic acid methylation is used for determining the prognosis of a subject suffering from prostate cancer.

The prognosis can be used by the clinician to determine the course of treatment, and to monitor the course of treatment. As is understood by the skilled practitioner, prognosis is a prediction and can change during the course of treatment.

### V. Samples

Samples for use in the methods of the invention include cells or tissues obtained from any solid tumor, samples taken from blood, blood plasma, serum, cells, a cellular extract, a cellular aspirate, tissues, a tissue sample, or a tissue biopsy. Tumor DNA can be found in various body fluids and these fluids can potentially serve as diagnostic material.

Any nucleic acid specimen, in purified or nonpurified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, such as contained in whole human DNA.

The nucleic acid-containing sample or specimen used for detection of methylated CpG may be extracted by a variety of techniques such as that described by Maniatis, et al. (Molecular Cloning: A Laboratory Manual, Cold Spring Harbor, N.Y., pp 280, 281, 1982).

If the extracted sample is impure (e.g., plasma, serum, stool, ejaculate, sputum, saliva, ductal cells, nipple aspiration fluid, ductal lavage fluid, cerebrospinal fluid or blood or a sample embedded in parrafin), it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily

Preferably, the method of amplifying is by PCR, as described herein and as is commonly used by those of ordinary skill in the art. However, alternative methods of amplification have been described and can also be employed. PCR techniques and many variations of PCR are known. Basic PCR techniques are described by Saiki et al. (1988 Science 239:487-491) and by U.S. Pat. Nos. 4,683,195, 4,683,202 and 4,800,159, each of which is incorporated herein by reference.

The conditions generally required for PCR include temperature, salt, cation, pH and related conditions needed for efficient copying of the master-cut fragment. PCR conditions include repeated cycles of heat denaturation (i.e. heating to at least about 95 C.) and incubation at a temperature permitting primer: adaptor hybridization and copying of the master-cut DNA fragment by the amplification enzyme. Heat stable amplification enzymes like the pwo, Thermus aquaticus or Thermococcus litoralis DNA polymerases which eliminate the need to add enzyme after each denaturation cycle, are commercially available. The salt, cation, pH and related factors needed for enzymatic amplification activity are available from commercial manufacturers of amplification enzymes.

As provided herein an amplification enzyme is any enzyme which can be used for in vitro nucleic acid amplification, e.g. by the above-described procedures. Such amplification enzymes include pwo, Escherichia coli DNA polymerase I, Klenow fragment of E. coli polymerase I, T4 DNA polymerase, T7 DNA polymerase, Thermus aquaticus (Taq) DNA polymerase, Thermococcus litoralis DNA polymerase, SP6 RNA polymerase, T7 RNA polymerase, T3 RNA polymerase, T4 polynucleotide kinase, Avian Myeloblastosis Virus reverse transcriptase, Moloney Murine Leukemia Virus reverse transcriptase, T4 DNA ligase, E. coli DNA ligase or Q.beta. replicase. Preferred amplification enzymes are the pwo and Taq polymerases. The pwo enzyme is especially preferred because of its fidelity in replicating DNA.

Once amplified, the nucleic acid can be attached to a solid support, such as a membrane, and can be hybridized with any probe of interest, to detect any nucleic acid sequence. Several membranes are known to one of skill in the art for the adhesion of nucleic acid sequences. Specific non-limiting examples of these membranes include nitrocellulose (NITROPURE) or other membranes used in for detection of gene expression such as polyvinylchloride, diazotized paper and other commercially available membranes such as GENESCREEN, ZETAPROBE. (Biorad), and NYTRAN. Methods for attaching nucleic acids to these membranes are well known to one of skill in the art. Alternatively, screening can be done in a liquid phase.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2.times.SSC/0.1% SDS at about room temperature (hybridization conditions); 0.2.times.SSC/0.1% SDS at about room temperature (low stringency conditions); 0.2.times.SSC/0.1% SDS at about 42.degree. C. (moderate stringency conditions); and 0.1.times.SSC at about 68.degree. C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically. In general, conditions of high stringency are used for the hybridization of the probe of interest.

The probe of interest can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the probe, or will be able to ascertain such, using routine experimentation.

### VI. Kits

The methods of the invention are ideally suited for the preparation of kits.

The invention features kits for identifying the nucleic acid methylation state of one or more genes comprising gene specific primers for use in polymerase chain reaction (PCR), and instructions for use.

The invention also features kits for detecting prostate cancer by detecting nucleic acid methylation of one or more genes, the kit comprising gene specific primers for use in polymerase chain reaction (PCR), and instructions for use.

As described above, the PCR, in particularly preferred examples, is methylation specific PCR (MSP).

In certain embodiments, any gene comprising one or more CpG islands in the promoter region can be detected using the kits of the invention. In certain preferred examples, the one or more genes are selected from ASC and CDH13.

The kits can be used to detect methylation of at least one of the genes as described herein. In some examples, can be used to detect methylation of at least two of the genes as described herein.

Carrier means are suited for containing one or more container means such as vials, tubes, and the like, each of the container means comprising one of the separate elements to be used in the method. In view of the description provided herein of invention methods, those of skill in the art can readily determine the apportionment of the necessary reagents among the container means. For example, one of the container means can comprise a container containing gene specific primers for use in polymerase chain reaction methods of the invention. In addition, one or more container means can also be included which comprise a methylation sensitive restriction endonuclease.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### Examples

Biochemical (PSA) recurrence of prostate cancer following radical prostatectomy remains a major problem. Better biomarkers are needed to identify high-risk patients. DNA methylation of promoter regions leads to gene silencing in many cancers. The experiments descriebd herein assess the impact of DNA methylation on the identification of recurrent prostate cancer.

### Example

The DNA methylation status of fifteen genes, whose loss of expression is involved in the progression of cancer and for which there is either data to support, or insufficient evidence to exclude, DNA methylation as a cause of reduced gene expression, was first determined. The results are shown in Table 1, below. Table 1 shows the biological importance and methylation frequency of the genes shown in the panel.

TABLE 1

| | BIOLOGICAL | METHYLATION |
|---|---|---|
| GENE | IMPORTANCE | FREQUENCY (%) |
| GSTP1 | Protects against oxidant stress | 60 |
| | and genomic damage | |
| MGMT | Involved in DNA repair; | 30 |
| | Methylation correlates with | |
| | increased responsiveness to | |
| | alkylating agents | |
| ASC | Activates procaspase-1 and | 37 |
| | enhances drug-induced | |
| | apoptosis | |
| CDKNZA | Cyclin-dependent kinase | 30 |
| | inhibitor and G1-S checkpoint | |
| | regulator | |
| *CHFR | G2-M checkpoint regulator; | 0 |
| | Loss of expression leads to | |
| | enhanced sensitivity to [symbol] | |
| EDNRB | Thought to mediate clearance | 15 |
| | of endothelin, a potent mitogen | |
| | of prostate cancer cells and | |
| | stimulator of bony metastasis | |
| *AR | Receptor for testosterone and | 0 |
| | dihydrotestosterone, which | |
| | stimulate prostate cancer | |
| | growth | |
| *PTEN | Tumor suppressor gene, which | 0 |
| | regulates G1-S transition; | |
| | inhibits Akt/Pl-3 kinase | |
| | pathway | |
| *CDH1 | Cell-cell adhesion protein | 0 |
| | whose loss correlates with | |
| | invasiveness; Repression | |
| | leads to epithelial- | |
| | mesenchymal transition | |
| CDH13 | Cell-cell adhesion protein | 45 |
| | whose loss correlates with | |
| | invasiveness | |
| CD44 | Membrane phosphoprotein | 19 |
| | Important in tumor-matrix | |
| | interactions; Down regulation | |
| | leads to increased risk of | |
| | metastasis | |
| TIMP3 | inhibitor of matrix | 4 |
| | metalloproteinases, which | |
| | promote issue invasion | |
| RUNX3 | Downstream effector of TGF-B | 44 |
| | pathway | |
| APC | Inhibitor of Wnt pathway | 71 |
| | signaling | |
| WIF-1 | Inhibitor of Wnt pathway | 28 |
| | signaling | |

*Only 50/151 samples analyzed by MSP [symbol]
indicates data missing or illegible when filed

The frequency of methylation for each gene is shown in Table 1. Methylation was not detected for any of the CDH1 (cadherin 1), PTEN (phosphatase and tensin homolog), CHFR (checkpoint with forkhead and ring finger domains), and AR

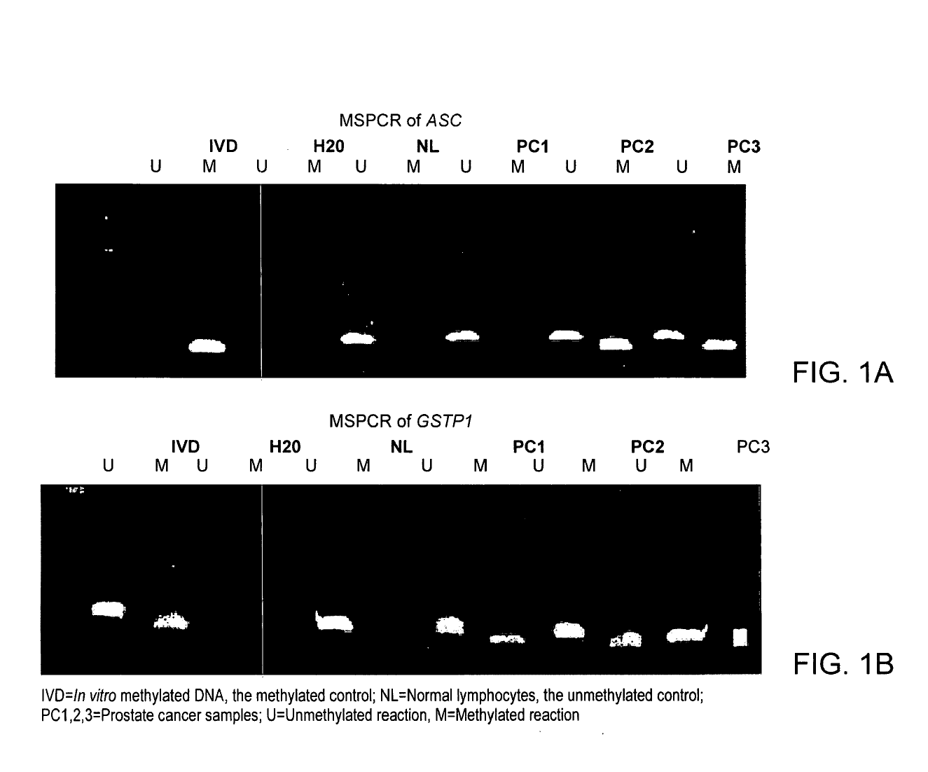

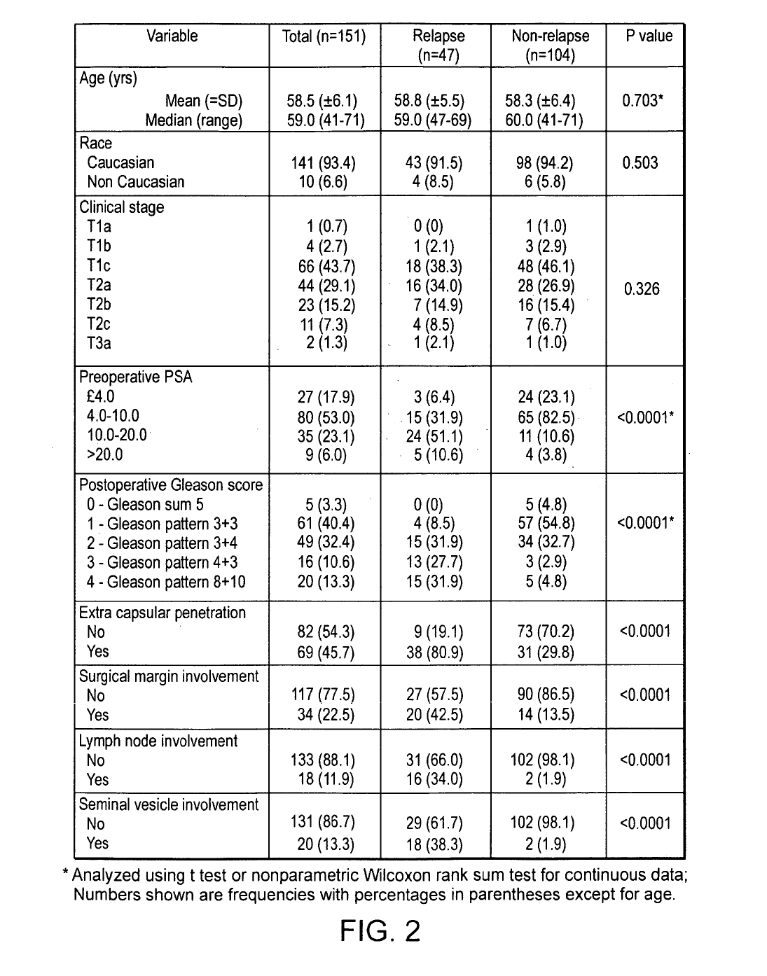

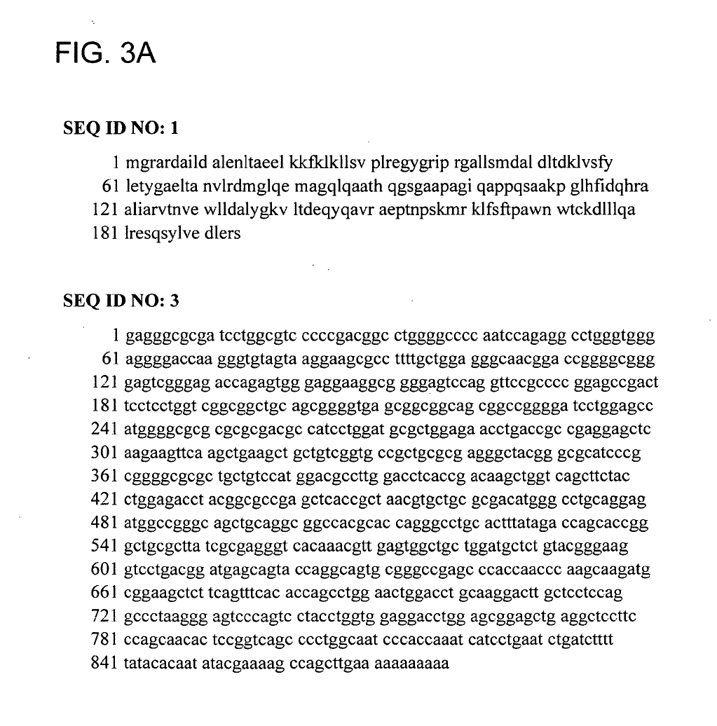

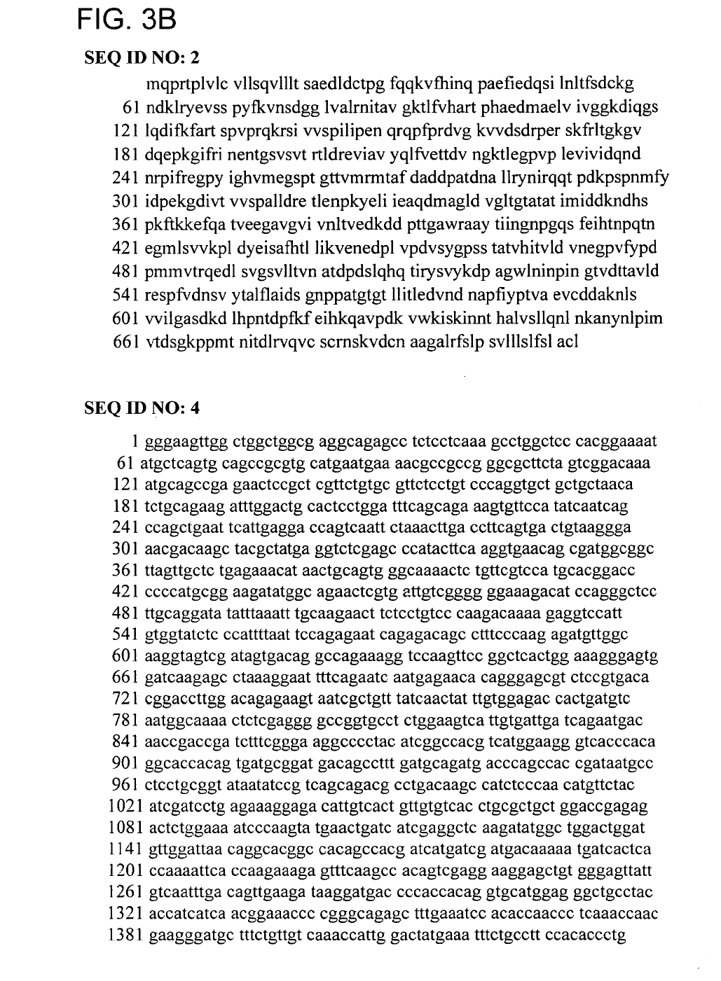

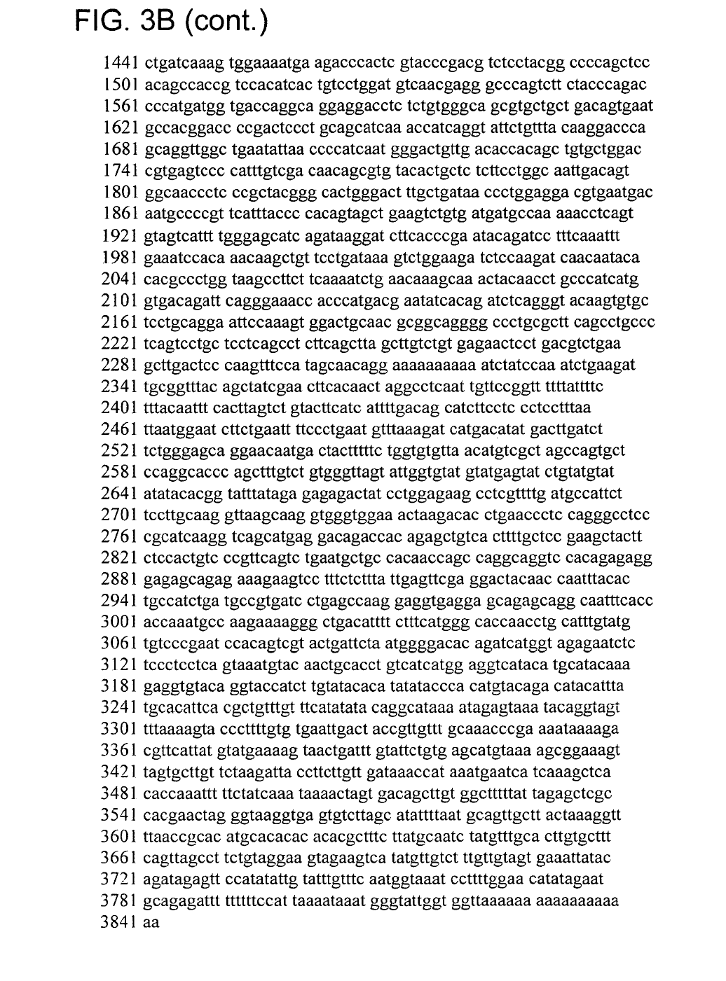

(Androgen Receptor) genes after studying one-third of the tumors, and therefore no further samples were tested. For the other 11 genes, all 151 samples were evaluated. Overall, 99% of the PCR reactions were successful and informative. Representative gels for ASC (Apoptosis-associated speck-like protein containing a CARD) and GSTP1 (glutathione S-transferase 1) are shown in FIGS. 1A and B. The demographic, clinical and pathologic characteristics for the 151 patients is shown in the Table in FIG. 2.

Methylation of CDKN2A was associated with a statistically significant decreased risk of biochemical recurrence with an odds ratio of 0.43 (95% CI=0.19-0.98; P=0.046) in univariate analysis only. Although the methylation of no single gene was found to be associated with a statistically significant increased risk of recurrence in univariate analysis, trends existed with marginal significance for ASC (OR=1.64; 95% CI=0.81-3.32; P=0.171) and CDH13 (OR=1.80; 95% CI=0.90-3.61; P=0.099). Next, a methylation profile of combining these 2 genes (ASC and CDH13) was analyzed to explore its ability to predict the probability of a biochemical recurrence. Tumors with methylation of ASC and/or CDH13 were associated with an increased risk of recurrence compared to tumors without methylation of both of these genes in both univariate (OR=2.42; 95% CI=1.45-5.11; P=0.021) and multivariable analyses (OR=5.64; 95% CI=1.47-21.7; P=0.012) after adjusting for pre-operative PSA, post-operative Gleason score, extra capsular penetration, and involvement of the lymph nodes, seminal vesicles, and surgical margins. This is shown in Table 2, below.

TABLE 2
DNA Methylation is Associated with an Increased Risk
of PSA Recurrence
A. Univariate Analysis of the Risk of Biochemical Recurrence

| Variable | Odds ratio | 95% CI* | P value |
|---|---|---|---|
| Preoperative PSA (continuous) | 1.15 | 1.07-1.24 | 0.0001 |
| Postoperative Gleason score | 4.05 | 2.55-6.44 | <0.0001 |
| (continuous) | | | |
| Extra capsular penetration | 9.67 | 4.17-22.4 | <0.0001 |
| (yes vs. no) | | | |
| Lymph node involvement (yes vs. no) | 26.8 | 5.62-118.5 | <0.0001 |
| Seminal vesicle involvement | 31.0 | 6.80-141.6 | <0.0001 |
| (yes vs. no) | | | |
| Surgical margin involvement | 4.86 | 2.08-10.4 | 0.0002 |
| (yes vs. no) | | | |
| ASC. CDH13 (at least one vs. | 2.42 | 1.45-5.11 | 0.021 |
| none methylated) | | | |

B. Multivariable Analysis of the Risk of Biochemical Recurrence

| Covariate | Odds ratio | 95% CI* | P value |
|---|---|---|---|
| Preoperative PSA (continuous) | 1.00 | 0.91-1.09 | 0.988 |
| Postoperative Gleason score | 3.20 | 1.70-6.01 | 0.0003 |
| (continuous) | | | |
| Extra capsular penetration | 3.45 | 1.09-10.9 | 0.036 |
| (yes vs. no) | | | |
| Lymph node involvement (yes vs. no) | 6.47 | 1.05-40.0 | 0.045 |
| Seminal vesicle involvement | 12.1 | 1.75-83.4 | 0.012 |
| (yes vs. no) | | | |
| Surgical margin involvement | 6.95 | 1.94-24.9 | 0.003 |
| (yes vs. no) | | | |
| ASC, CDH13 (at least one vs. | 5.84 | 1.47-21.7 | 0.012 |
| none methylated) | | | |

*CI: Confidence interval.

The operating characteristics of combining the two genes were further evaluated and compared with the previously defined Rw score (5). A high Rw′ score, as defined by a value >2.84, was more specific and was associated with a higher positive predictive value and higher likelihood ratio for recurrence than methylation of ASC in combination with CDH13 (Table 3, below)(5). However, a trend toward a higher sensitivity of the methylation of ASC in combination with CDH13 (72.3%; 95% C1=57.4-84.4%) compared to the score (55.3%; 95% C1=40.1-69.8%) was observed although it did not reach statistical significance (P=0.102). Additionally, the methylation status of these 2 genes had a negative predictive value of 79% (95% C1=66.8-88.3%), which was not significantly different from the Rw' score (82.2%; 95% C1=74.1-88.6%) (P=0.767) and negative likelihood ratio of 0.58 (95% CI=0.35-0.95) with no significant difference from the Rw' score (Table 3).

TABLE 3

| | *Sensitivity (%) | ^{§}Specificity (%) | ^{¥}PPV (%) | ^{†}NPV (%) | ^{ε}LR+ | ^{‡}LR− |
|---|---|---|---|---|---|---|
| Variable | (95% CI^{£}) | (95% CI) | (95% CI) | (95% CI) | (95% CI) | (95% CI) |
| ^{#}R_{w}′ | 55.3 | 96.1 | 83.9 | 82.2 | 11.3 | 0.5 |
| (>2.84) | (40.1-69.8) | (88.9-98.4) | (68.3-94.5) | (74.1-88.6) | (4.6-27.5) | (0.3-0.6) |
| ASC, CDH13 | 72.3 | 48.0 | 39.1 | 78.0 | 1.4 | 0.6 |
| (at least 1 methylated) | (57.4-84.4) | (38.0-58.2) | (28.8-50.1) | (86.8-88.3) | (1.1-1.8) | (0.3-0.9) |

^{#}R_{w}′, weighted risk of recurrence, based on lymph node status, seminal vesicle status, surgical margin status, and postoperative Gleason score
*Sensitivity is calculated as the number of true positives divided by the number of true positives plus false negatives
^{§}Specificity is calculated as the number of true negatives divided by the number of true negatives plus false positives
^{¥}PPV (positive predictive value) is calculated as the number of true positives divided by the number of true positives plus false positives
^{†}NPV (negative predictive value) is calculated as the number of true negatives divided by the\number of true negatives plus false negatives
^{ε}LR+ (likelihood ratio of a positive test result) is calculated as sensitivity/(1 − specificity)
^{‡}LR− (likelihood ratio of a negative test result) is calculated as (1 − sensitivity)/specificity.
^{£}CI: Confidence interval.

A pathology report after radical prostatectomy gives the clinician and patient valuable information about the risk of recurrence, but there are limitations. For the growing group of patients with early stage and low-grade prostate cancer, the outcome can be variable with some patients experiencing recurrences, some of which will be lethal. In addition, there can be significant inter-pathologist variability in the interpretation of the Gleason score, which is a powerful prognostic factor in prostate cancer(39).

There are two reports, both of which used quantitative MSP, which show that tumors with more than the median of methylation of PTGS2 or the highest quartile of methylation of APC, are independently associated with an increased risk of PSA recurrence or a reduced time to recurrence, respectively(34, 40). These two genes were methylated in the vast majority of prostate cancer samples analyzed in these studies. Quantitative MSP approaches do not adjust for the percentage of tumors cells which comprise a sample since a housekeeping gene, which is found in both normal and tumor cells such as GAPDH, is used as a control for the amount of total input DNA (tumor DNA+non-tumor DNA) in the assay. Since each tumor cell has 2 alleles of a given gene and since most methylated genes have bi-allelic DNA methylation, “more” methylation of a gene measured by a quantitative approach likely reflects a sample with more tumors cells. From the prostate cancer pathology literature, it is clear that higher tumor burden, as measured by involvement of multiple cores or a high percentage of tumor cells within each core, is associated with prostate cancer aggressiveness(41).

In this study, DNA methylation was not present in the promoter of several genes including CHFR, CDH1, PTEN, and AR after analyzing 50 samples. CHFR was evaluated in these studies because its methylation correlates with sensitivity to taxane-based chemotherapy, and given the efficacy of docetaxel in prostate cancer(30, 42-44). It appears that alternative mechanisms besides DNA methylation of CHFR are responsible for taxane-sensitivity in prostate cancer. There are several reports of methylation of the cell adhesion gene CDH1 in prostate cancer cell lines, and loss of expression of this protein is seen in more aggressive prostate cancers(29, 45). In human tumors, methylation of CDHI was not found, which is consistent with an earlier report(34). PTEN transcript was found to be reactivated by treatment with the DNA methyltransferase inhibitor, 5-aza-2′-deoxycytadine, in a prostate cancer xenograft(24). However, methylation of this gene was not detected in 50 samples. It is still possible that PTEN is silenced through epigenetic mechanisms, including histone methylation or DNA methylation of a different region. Finally, methylation of the AR was not detected, which has mainly been described in vitro rather than in human prostate cancer samples(31).

For several genes, the methylation frequency that was reported herein is different than the published literature. Much of this difference is likely due to study design, in which the numbers of patients with the outcomes of interest, recurrence versus non-recurrence, were pre-determined. For CDKN2A, a methylation frequency of 30% was found, which is higher than previous reports, which range from 2-6% likely due to the more sensitive nested PCR approach and the study design used herein (16, 34). There are conflicting reports about the role of CDKN2A in prostate cancer, but up-regulation appears to be more common than loss of expression and is associated with an increased risk of recurrence(46). Finally, for ASC, a methylation frequency of 37% was found, which is lower than a previous report, which found a methylation frequency of 65%(47). In that series, methylation of ASC in the normal-appearing epithelium of prostate cancer patients, but not in the tumors, was associated with an increased risk of biochemical recurrence. The observed differences between that report and the results reported herein may relate to the number of PCR cycles used and the sensitivity of each approach and the study. The methylation of individual genes in the instant panel was not associated with a statistically significant increased risk of recurrence. Given the low frequency of methylation for some of the genes examined herein, it is possible that the cohort size did not allow for adequate determination of positive associations. From a biological standpoint, many of the genes which were studied may be more important for early steps in tumor formation rather than for metastasis. Alternatively, lack of methylation of some genes may be a surrogate for inactivation of other genes in overlapping pathways, which have a greater role in conferring invasiveness.

Tumors with methylation of either ASC and/or CDH13 were associated with an increased risk of recurrence versus tumors without methylation of these genes. ASC, Apoptosis-associated speck-like protein containing a CARD, was first identified in human leukemia, and this protein was found to form aggregates, which appeared like a “speck” during apoptosis(48). This same group noted that this protein contains a CARD, caspase recruitment domain, which is common to certain proteins involved in apoptosis. Knockdown of ASC in leukemia cell lines results in impaired apoptosis in response to etoposide(48). Methylation of ASC, also known as TMS-1, is common in breast cancer, and ASC, when expressed, appears to effect apoptosis via the caspases(26).

CDH13 is a membrane of the class of glycoproteins called cadherins, which are calcium-dependent cell-cell adhesion molecules(49). Members of this protein family are commonly lost in solid tumors, and their loss may be important for epithelial to mesenchymal transition and conference of increased metastatic potential(50, 51). Expression of CDH1, which is another member of this family, is commonly diminished in prostate cancer, although the mechanism does not appear to be DNA methylation. CDH13 loss via DNA methylation is a common event in prostate cancer and is associated with tumors with Gleason scores greater than or equal to 7(16).

Tumors lacking methylation of both of these genes are associated with a much lower risk of recurrence than tumors with methylation of these genes. While the functions of these genes are disparate, they each play key roles in suppressing an aggressive phenotype. A prostate tumor with loss of expression of ASC would be more likely to survive under conditions of DNA damage, which would normally induce apoptosis. The expected result would be a cancer cell with genetic instability and resistance to DNA damage-inducing agents and other cellular stressors. Loss of CDH13, on the other hand, might allow a prostate cancer cell to grow in an anchorage-independent manner and allow for ease of dissemination via the lymphatic channels or blood vessels.

The multivariable analysis showed that methylation of ASC and/or CDH13 was independently associated with an increased risk of recurrence (OR=5.64; 95% CI 1.39-18.2; P=0.012) after adjusting for pre-operative PSA, modified Gleason score, extra capsular penetration, and involvement of the lymph nodes, seminal vesicles, or lymph nodes. The 2-gene methylation marker showed a trend toward a higher sensitivity for detecting recurrences than the Ry; score (Table 3; P=0.102), which is already in use in the clinic and which has been used to stratify patients at high risk of recurrence to an adjuvant Phase 2 clinical trial with docetaxel(52). The lack of statistical significance may have resulted from the limited sample size (n=47) of the recurrent patients. However, the real value of these methylation findings emerges when one examines the patients who recurred. Thirty-four percent (95% C1=21-49%) of the men who recurred were classified as low risk due to R,' scores <2.84, but samples from these same men were appropriately identified as recurrences by the methylation status of ASC or CDH13. These findings suggest that determination of methylation of these genes may have the greatest utility for those patients treated with surgery whose tumors lack adverse clinico-pathological features but who are still certainly at risk of recurrence, and also for patients treated with radiotherapy, for whom involvement of the seminal vesicle, surgical margin, capsule, and lymph nodes is generally not available and for whom greater recurrence predictors are clearly needed.

This study of the DNA methylation patterns of 151 patients with localized prostate cancer undergoing radical prostatectomy is unique and important for several reasons. First, it is largest known series in which DNA methylation was found to be independently associated with an increased risk of biochemical recurrence even when all of the currently accepted clinico-pathological variables were incorporated into a multivariable analysis. Our DNA methylation marker comprised of ASC and CDH13 appeared to have added value to the clinico-pathological variables since one-third of the recurrences, which were not identified as high risk by the previously validated Rw score, harbored these DNA methylation changes. The cohort examined here included a heterogeneous group of patients with tumors with a variety of Gleason scores, pre-operative PSA values, and pathological stages. The known clinico-pathologic variables previously associated with an increased risk of recurrence were all still predictive in the group of patients examined, highlighting the representativeness of this cohort. A pre-defined binary endpoint was used (the presence of a band in the methylated reaction was scored as a methylated sample) rather than utilizing medians or quartiles, which are not applicable to other groups of patients. The MSP assay was reliable and worked 99% of the time on the archived, paraffin specimens studied, the vast majority of which were over ten years old.

In conclusion, the finding that the methylation status of 2 genes from the panel, ASC and CDH13, are related to recurrence in patients undergoing radical prostatectomy could have great importance. Given the high sensitivity, high negative predictive value, and low negative likelihood ratio, tumors without DNA methylation of ASC and CDH13 were associated with a significantly reduced risk of recurrence after radical prostatectomy.

### Methods

The invention was performed using, but not limited to, the methods as described herein.

Study Population

Overall, the rate of biochemical recurrence at 5 years after radical prostatectomy ranges from 16 to 31 percent(5, 35, 36). Using a retrospective, nested case-control design, we identified 151 patients with at least 5 years of follow-up after surgery. One hundred and four patients, or two-thirds, were without biochemical recurrence, and 47, or one-third, had experienced a biochemical recurrence. All patients had undergone a radical prostatectomy at our institution after the year 1988 when PSA testing was routine. The patient characteristics are shown in Table 2. Our primary endpoint was biochemical recurrence-free survival as defined by a PSA 0.2 ng/mL at five years post-surgery. This was equivalent to one of our secondary endpoint, disease-free survival, since all patients who recurred had PSA values>0.2 ng/mL. The investigator performing all the PCR reactions (JJA) was blinded to the clinico-pathological data of all the patients.

Tissue Samples

After Institutional Review Board permission was granted, 151 paraffin-embedded prostate cancer samples from radical prostatectomies were mounted on H&E slides, and 4 1 Opm sections representing the highest Gleason score were taken from each sample. Specimens were deparaffinized with xylene and washed twice with 100% ethanol. The samples were digested, with Proteinase K, and DNA was extracted with phenol-chloroform as described previously(37).

Nested Methylation Specific PCR

Nested MSP was performed as described previously(38). In vitro methylated DNA and peripheral blood lymphocytes from a normal volunteer, which were bisulfite-modified, served as methylated and unmethylated controls. All reactions also had a negative H2O control. The PCR products were loaded onto 2.5% agarose gels stained with GelStar (Cambrex, E. Rutherford, N.J.) and subjected to electrophoresis. The products were subsequently diluted 1:500 in H2O and served as the template for the second round PCR reaction with both unmethylated and methylated primers, respectively. All samples were run on 2.5% agarose gels as above. The presence of a methylated band was scored as a methylated sample. Samples with only an unmethylated band were scored as unmethylated. Samples, which contained neither band, were scored as non-evaluable. Primer sequences and PCR conditions are available in Supplementary Table 1.

Statistical Analysis

The absence of a PSA>0.2ng/mL at 5 years post-surgery was used to define a dichotomous outcome of biochemical recurrence. DNA methylation was treated as a binary variable (presence versus absence of methylation). Student's t test or its nonparametric alternative was used to analyze continuous data and Fisher's exact test was used for categorical data. The associations of gene methylation with biochemical recurrence were first assessed in univariate logistic regression models and then in a multivariable logistic regression model adjusting for the modified post-operative Gleason score and pre-operative PSA, which were treated as continuous variables, and whether the capsule, lymph nodes, seminal vesicles, or surgical margins were involved. Odds ratios and 95% confidence intervals were reported. Operating characteristics of selected gene methylation and R.,' score (a cutoff of 2.84 was used for risk stratification as defined previously in detection of biochemical recurrence) were summarized using sensitivity, specificity, positive and negative predictive values, and likelihood ratios(5). McNemar's test was used to compare paired proportions. All statistical tests were two-sided with p values 0.05 considered statistically significant. Analyses were performed with SAS software (Version 9.1, Cary, N.C.).

The present invention has been described in detail, including the preferred embodiments thereof. However, it will be appreciated that those skilled in the art, upon consideration of the present disclosure, may make modifications and/or improvements of this invention and still be within the scope and spirit of this invention as set forth in the following claims.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### REFERENCES

1. American Cancer Society. Cancer Facts and Figures., 2004.
2. Catalona, W. J., Smith, D. S., Ratliff, T. L., and Basler, J. W. Detection of organ-confined prostate cancer is increased through prostate-specific antigen-based screening. Jama, 270: 948-954, 1993.
3. Brawer, M. K., Chetner, M. P., Beatie, J., Buchner, D. M., Vessella, R. L., and Lange, P. H. Screening for prostatic carcinoma with prostate specific antigen. J Urol, 147: 841-845, 1992.
4. Pound, C. R., Partin, A. W., Eisenberger, M. A., Chan, D. W., Pearson, J. D., and Walsh, P. C. Natural history of progression after PSA elevation following radical prostatectomy. Jama, 281: 1591-1597, 1999.
5. Roberts, W. W., Bergstralh, E. J., Blute, M. L., Slezak, J. M., Carducci, M., Han, M., Epstein, J. I., Eisenberger, M. A., Walsh, P. C., and Partin, A. W. Contemporary identification of patients at high risk of early prostate cancer recurrence after radical retropubic prostatectomy. Urology, 57: 1033-1037, 2001.
6. Kaftan, M. W., Eastham, J. A., Stapleton, A. M., Wheeler, T. M., and Scardino, P. T. A preoperative nomogram for disease recurrence following radical prostatectomy for prostate cancer. J Natl Cancer Inst, 90: 766-771, 1998.
7. McMenamin, M. E., Soung, P., Perera, S., Kaplan, I., Loda, M., and Sellers, W. R. Loss of PTEN expression in paraffin-embedded primary prostate cancer correlates with high Gleason score and advanced stage. Cancer Res, 59: 4291-4296, 1999.
8. Cairns, P., Okami, K., Halachmi, S., Halachmi, N., Esteller, M., Herman, J. G., Jen, J., Isaacs, W. B., Bova, G. S., and Sidransky, D. Frequent inactivation of PTEN/MMAC1 in primary prostate cancer. Cancer Res, 57: 4997-5000, 1997.
9. Tomlins, S. A., Rhodes, D. R., Perner, S., Dhanasekaran, S. M., Mehra, R., Sun, X. W., Varambally, S., Cao, X., Tchinda, J., Kuefer, R., Lee, C., Montie, J. E., Shah, R. B., Pienta, K. J., Rubin, M. A., and Chinnaiyan, A. M. Recurrent fusion of TMPRSS2 and ETS transcription factor genes in prostate cancer. Science, 310: 644-648, 2005.
10. Bowen, C., Bubendorf, L., Voeller, H. J., Slack, R., Willi, N., Sauter, G., Gasser, T. C., Koivisto, P., Lack, E. E., Kononen, J., Kallioniemi, 0. P., and Gelmann, E. P. Loss of NKX3.1 expression in human prostate cancers correlates with tumor progression. Cancer Res, 60: 6111-6115, 2000.
11. Meeker, A. K., Hicks, J. L., Platz, E. A., March, G. E., Bennett, C. J., Delannoy, M. J., and De Marzo, A. M. Telomere shortening is an early somatic DNA alteration in human prostate tumorigenesis. Cancer Res, 62: 6405-6409, 2002.
12. Lee, W. H., Morton, R. A., Epstein, J. I., Brooks, J. D., Campbell, P. A., Bova, G. S., Hsieh, W. S., Isaacs, W. B., and Nelson, W. G. Cytidine methylation of regulatory sequences near the pi-class glutathione S-transferase gene accompanies human prostatic carcinogenesis. Proc Natl Acad Sci USA, 91: 11733-11737, 1994.
13. Herman, J. G., Merlo, A., Mao, L., Lapidus, R. G., Issa, J. P., Davidson, N. E., Sidransky, D., and Baylin, S. B. Inactivation of the CDKN2/p16/MTS1 gene is frequently associated with aberrant DNA methylation in all common human cancers. Cancer Res, 55: 4525-4530, 1995.
14. Kito, H., Suzuki, H., Ichikawa, T., Sekita, N., Kamiya, N., Akakura, K., Igarashi, T., Nakayama, T., Watanabe, M., Harigaya, K., and Ito, H. Hypermethylation of the CD44 gene is associated with progression and metastasis of human prostate cancer. Prostate, 49: 110-115, 2001.
15. Lou, W., Krill, D., Dhir, R., Becich, M. J., Dong, J. T., Frierson, H. F., Jr., Isaacs, W. B., Isaacs, J. T., and Gao, A. C. Methylation of the CD44 metastasis suppressor gene in human prostate cancer. Cancer Res, 59: 2329-2331, 1999.
16. Maruyama, R., Toyooka, S., Toyooka, K. 0., Virmani, A. K., Zochbauer-Muller, S., Farinas, A. J., Minna, J. D., McConnell, J., Frenkel, E. P., and Gazdar, A. F. Aberrant promoter methylation profile of prostate cancers and its relationship to clinicopathological features. Clin Cancer Res, 8: 514-519, 2002.
17. Nakayama, T., Watanabe, M., Yamanaka, M., Hirokawa, Y., Suzuki, H., Ito, H., Yatani, R., and Shiraishi, T. The role of epigenetic modifications in retinoic acid receptor beta2 gene expression in human prostate cancers. Lab Invest, 81: 1049-1057, 2001.
18. Chakravarti, A., Heydon, K, Wu, C. L., Hammond, E., Pollack, A., Roach, M., Wolkov, H., Okunieff, P., Cox, J., Fontanesi, J., Abrams, R., Pilepich, M., and Shipley, W. Loss of p16 expression is of prognostic significance in locally advanced prostate cancer: an analysis from the Radiation Therapy Oncology Group protocol 86-10. J Clin Oncol, 21: 3328-3334, 2003.
19. Nelson, J. B., Chan-Tack, K., Hedican, S. P., Magnuson, S. R., Opgenorth, T. J., Bova, G. S., and Simons, J. W. Endothelin-1 production and decreased endothelin B receptor expression in advanced prostate cancer. Cancer Res, 56: 663-668, 1996.
20. Nelson, J. B., Hedican, S. P., George, D. J., Reddi, A. H., Piantadosi, S., Eisenberger, M. A., and Simons, J. W. Identification of endothelin-1 in the pathophysiology of metastatic adenocarcinoma of the prostate. Nat Med, 1: 944-949, 1995.
21. Bohm, F., Pernow, J., Lindstrom, J., and Ahlborg, G. ETA receptors mediate vasoconstriction, whereas ETB receptors clear endothelin-1 in the splanchnic and renal circulation of healthy men. Clin Sci (Lond), 104: 143-151, 2003.
22. Nelson, J. B., Lee, W. H., Nguyen, S. H., Jarrard, D. F., Brooks, J. D., Magnuson, S. R., Opgenorth, T. J., Nelson, W. G., and Bova, G. S. Methylation of the 5′ CpG island of the endothelin B receptor gene is common in human prostate cancer. Cancer Res, 57: 35-37, 1997.
23. Yamanaka, M., Watanabe, M., Yamada, Y., Takagi, A., Murata, T., Takahashi, H., Suzuki, H., Ito, H., Tsukino, H., Katoh, T., Sugimura, Y., and Shiraishi, T. Altered methylation of multiple genes in carcinogenesis of the prostate. Int J Cancer, 106: 382-387, 2003.
24. Whang, Y. E., Wu, X., Suzuki, H., Reiter, R. E., Tran, C., Vessella, R. L., Said, J. W., Isaacs, W. B., and Sawyers, C. L. Inactivation of the tumor suppressor PTEN/MMAC 1 in advanced human prostate cancer through loss of expression. Proc Natl Acad Sci USA, 95: 5246-5250, 1998.
25. Stehlik, C., Krajewska, M., Welsh, K., Krajewski, S., Godzik, A., and Reed, J. C. The PAAD/PYRIN-only protein POP1/ASC2 is a modulator of ASC-mediated nuclear-factor-kappa B and pro-caspase-1 regulation. Biochem J, 373: 101-113, 2003.
26. Conway, K. E., McConnell, B. B., Bowring, C. E., Donald, C. D., Warren, S. T., and Vertino, P. M. TMS1, a novel proapoptotic caspase recruitment domain protein, is a target of methylation-induced gene silencing in human breast cancers. Cancer Res, 60: 6236-6242, 2000.
27. Wissmann, C., Wild, P. J., Kaiser, S., Roepcke, S., Stoehr, R., Woenckhaus, M., Kristiansen, G., Hsieh, J. C., Hofstaedter, F., Hartmann, A., Knuechel, R., Rosenthal, A., and Pilarsky, C. WIF1, a component of the Wnt pathway, is down-regulated in prostate, breast, lung, and bladder cancer. J Pathol, 201: 204-212, 2003.
28. Mazieres, J., He, B., You, L., Xu, Z., Lee, A. Y., Mikami, I., Reguart, N., Rosell, R., McCormick, F., and Jablons, D. M. Wnt inhibitory factor-1 is silenced by promoter hypermethylation in human lung cancer. Cancer Res, 64: 4717-4720, 2004.
29. Graff, J. R., Herman, J. G., Lapidus, R. G., Chopra, H., Xu, R., Jarrard, D. F., Isaacs, W. B., Pitha, P. M., Davidson, N. E., and Baylin, S. B. E-cadherin expression is silenced by DNA hypermethylation in human breast and prostate carcinomas. Cancer Res, 55: 5195-5199, 1995.
30. Toyota, M., Sasaki, Y., Satoh, A., Ogi, K., Kikuchi, T., Suzuki, H., Mita, H., Tanaka, N., Itoh, F., Issa, J. P., Jair, K. W., Schuebel, K. E., Imai, K., and Tokino, T. Epigenetic inactivation of CHFR in human tumors. Proc Natl Acad Sci USA, 100: 7818-7823, 2003.
31. Jarrard, D. F., Kinoshita, H., Shi, Y., Sandefur, C., Hoff, D., Meisner, L. F., Chang, C., Herman, J. G., Isaacs, W. B., and Nassif, N. Methylation of the androgen receptor promoter CpG island is associated with loss of androgen receptor expression in prostate cancer cells. Cancer Res, 58: 5310-5314, 1998.
32. Kinoshita, H., Shi, Y., Sandefur, C., Meisner, L. F., Chang, C., Choon, A., Reznikoff, C. R., Bova, G. S., Friedl, A., and Jarrard, D. F. Methylation of the androgen receptor minimal promoter silences transcription in human prostate cancer. Cancer Res, 60: 3623-3630, 2000.
33. Kang, G. H., Lee, S., Lee, H. J., and Hwang, K. S. Aberrant CpG island hypermethylation of multiple genes in prostate cancer and prostatic intraepithelial neoplasia. J Pathol, 202: 233-240, 2004.
34. Yegnasubramanian, S., Kowalski, J., Gonzalgo, M. L., Zahurak, M., Piantadosi, S., Walsh, P. C., Bova, G. S., De Marzo, A. M., Isaacs, W. B., and Nelson, W. G. Hypermethylation of CpG islands in primary and metastatic human prostate cancer. Cancer Res, 64: 1975-1986, 2004.
35. Catalona, W. J. and Smith, D. S. 5-year tumor recurrence rates after anatomical radical retropubic prostatectomy for prostate cancer. J Urol, 152: 1837-1842, 1994.
36. Trapasso, J. G., deKernion, J. B., Smith, R. B., and Dorey, F. The incidence and significance of detectable levels of serum prostate specific antigen after radical prostatectomy. J Urol, 152: 1821-1825, 1994.
37. Herman, J. G., Jen, J., Merlo, A., and Baylin, S. B. Hypermethylation-associated inactivation indicates a tumor suppressor role for p15INK4B. Cancer Res, 56: 722-727, 1996.
38. Guo, M., Ren, J., House, M. G., Qi, Y., Brock, M. V., and Herman, J. G. Accumulation of promoter methylation suggests epigenetic progression in squamous cell carcinoma of the esophagus. Clin Cancer Res, 12: 4515-4522, 2006.
39. Glaessgen, A., Hamberg, H., Pihl, C. G., Sundelin, B., Nilsson, B., and Egevad, L. Interobserver reproducibility of modified Gleason score in radical prostatectomy specimens. Virchows Arch, 445: 17-21, 2004.
40. Rosenbaum, E., Hogue, M. 0., Cohen, Y., Zahurak, M., Eisenberger, M. A., Epstein, J. I., Partin, A. W., and Sidransky, D. Promoter hypermethylation as an independent prognostic factor for relapse in patients with prostate cancer following radical prostatectomy. Clin Cancer Res, 11: 8321-8325, 2005.
41. Epstein, J. I., Chan, D. W., Sokoll, L. J., Walsh, P. C., Cox, J. L., Rittenhouse, H., Wolfert, R., and Carter, H. B. Nonpalpable stage Tlc prostate cancer: prediction of insignificant disease using free/total prostate specific antigen levels and needle biopsy findings. J Urol, 160: 2407-2411, 1998.
42. Satoh, A., Toyota, M., Itoh, F., Sasaki, Y., Suzuki, H., Ogi, K., Kikuchi, T., Mita, H., Yamashita, T., Kojima, T., Kusano, M., Fujita, M., Hosokawa, M., Endo, T., Tokino, T., and !mai, K. Epigenetic inactivation of CHFR and sensitivity to microtubule inhibitors in gastric cancer. Cancer Res, 63: 8606-8613, 2003.
43. Tannock, I. F., de Wit, R., Berry, W. R., Horti, J., Pluzanska, A., Chi, K. N., Oudard, S., Theodore, C., James, N. D., Turesson, I., Rosenthal, M. A., and Eisenberger, M. A. Docetaxel plus prednisone or mitoxantrone plus prednisone for advanced prostate cancer. N Engl J Med, 351: 1502-1512, 2004.
44. Petrylak, D. P., Tangen, C. M., Hussain, M. H., Lara, P. N., Jr., Jones, J. A., Taplin, M. E., Burch, P. A., Berry, D., Moinpour, C., Kohli, M., Benson, M. C., Small, E. J., Raghavan, D., and Crawford, E. D. Docetaxel and estramustine compared with mitoxantrone and prednisone for advanced refractory prostate cancer. N Engi J Med, 351: 1513-1520, 2004.
45. Umbas, R., Isaacs, W. B., Bringuier, P. P., Schaafsma, H. E., Karthaus, H. F., Oosterhof, G. 0., Debruyne, F. M., and Schalken, J. A. Decreased E-cadherin expression is associated with poor prognosis in patients with prostate cancer. Cancer Res, 54: 3929-3933, 1994.
46. Jarrard, D. F., Modder, J., Fadden, P., Fu, V., Sebree, L., Heisey, D., Schwarze, S. R., and Friedl, A. Alterations in the p16/pRb cell cycle checkpoint occur commonly in primary and metastatic human prostate cancer. Cancer Lett, 185: 191-199, 2002.
47. Collard, R. L., Harya, N. S., Monzon, F. A., Maier, C. E., and O'Keefe, D. S. Methylation of the ASC gene promoter is associated with aggressive prostate cancer. Prostate, 66: 687-695, 2006.
48. Masumoto, J., Taniguchi, S., Ayukawa, K., Sarvotham, H., Kishino, T., Niikawa, N., Hidaka, E., Katsuyama, T., Higuchi, T., and Sagara, J. ASC, a novel 22-kDa protein, aggregates during apoptosis of human promyelocytic leukemia HL-60 cells. J Biol Chem, 274: 33835-33838, 1999.
49. Takeichi, M. Cadherin cell adhesion receptors as a morphogenetic regulator. Science, 251: 1451-1455, 1991.
50. Lee, S. W. H-cadherin, a novel cadherin with growth inhibitory functions and diminished expression in human breast cancer. Nat Med, 2: 776-782, 1996.
51. Toyooka, S., Toyooka, K. 0., Harada, K., Miyajima, K., Makarla, P., Sathyanarayana, U. G., Yin, J., Sato, F., Shivapurkar, N., Meltzer, S. J., and Gazdar, A. F. Aberrant methylation of the CDH13 (H-cadherin) promoter region in colorectal cancers and adenomas. Cancer Res, 62: 3382-3386, 2002.
52. Rosebaum E., Kibel A., Roth B., Wilding G., Dreicer R., Chatta G., Petrylak D., Dipaula R., Beer T., Eisenberger M. A pilot multicenter phase II study of adjuvant docetaxel for high risk prostate cancer patients after radical prostatectomy: preliminary data. Proc Am Soc Clin Oncol, 23: Abstract 4565, 2005.

1. A method for identifying a risk of developing prostate cancer in a subject comprising: detecting nucleic acid methylation of one or more genes in one or more samples, wherein detecting nucleic acid methylation identifies a risk of developing prostate cancer.

2. The method of claim 1, wherein the sample is one or more of blood, blood plasma, serum, cells, a cellular extract, a cellular aspirate, tissues, a tissue sample, or a tissue biopsy.

3. The method of claim 1, wherein the sample is taken from the prostate.

4. The method of claim 1, wherein the one or more genes comprise one or more CpG islands.

5. The method of claim 1, wherein the one or more genes is selected from ASC (Apoptosis-associated speck-like protein containing a CARD) or CDH13 (Cadherin 13).

6. The method of claim 1, wherein methylation of at least one of the genes is detected.

7. The method of claim 1, wherein methylation of at least two of the genes is detected.

8. The method of claim 6, wherein at least one of the genes is ASC.

9. The method of claim 6, wherein at least one of the genes is CDH13.

10. The method of claim 7, wherein at least two of the genes are ASC and CDH13.

11. A method for identifying a risk of developing prostate cancer in a subject comprising: detecting nucleic acid methylation of at least one or more genes in a sample, wherein the genes are selected from ASC or CDH13, and wherein detecting nucleic acid methylation identifies a risk of developing prostate cancer.

12. The method of claim 11, wherein at least one of the genes is ASC.

13. The method of claim 11, wherein at least one of the genes is CDH13.

14-17. (canceled)

18. The method of claim 1 or 11, wherein the subject has previously been diagnosed with prostate cancer.

19-23. (canceled)

24. A method for detecting or diagnosing prostate cancer in a subject comprising: detecting nucleic acid methylation of one or more genes in one or more samples, wherein detecting nucleic acid methylation is used to detect or diagnose prostate cancer, or
a method for predicting the recurrence of prostate cancer in a subject comprising: detecting nucleic acid methylation of one or more genes wherein detecting nucleic acid methylation of one or more genes is a predictor of the recurrence of prostate cancer.

25-29. (canceled)

30. A method for determining the prognosis of a subject suffering from prostate cancer comprising: detecting nucleic acid methylation of one or more genes wherein the detection of nucleic acid methylation is used for determining the prognosis of a subject suffering from prostate cancer.

31-37. (canceled)

38. A method for detecting or diagnosing prostate cancer in a subject comprising:
extracting nucleic acid from one or more cell or tissue samples;
detecting nucleic acid methylation of one or more genes in the sample; and
identifying the nucleic acid methylation state of one or more genes,
wherein nucleic acid methylation of genes indicates prostate cancer, or
a method for predicting the recurrence of prostate cancer in a subject comprising:
extracting nucleic acid from one or more cell or tissue samples;
detecting nucleic acid methylation of one or more genes in the sample; and
identifying the nucleic acid methylation state of one or more genes,
wherein nucleic acid methylation of genes is indicative of the recurrence of prostate cancer.

39-45. (canceled)

46. A method of treating a subject having or at risk for having prostate cancer comprising:
identifying nucleic acid methylation of one or more genes, where nucleic acid methylation indicates having or a risk for having prostate cancer; and
administering to the subject a therapeutically effective amount of a demethylating agent,
thereby treating a subject having or at risk for having prostate cancer.

47-68. (canceled)

69. A kit for identifying the nucleic acid methylation state of one or more genes comprising gene specific primers for use in polymerase chain reaction (PCR), and instructions for use, or
a kit for detecting prostate cancer by detecting nucleic acid methylation of one or more genes, the kit comprising gene specific primers for use in polymerase chain reaction (PCR), and instructions for use.

70-77. (canceled)
